(19) United States
(12) Patent Application Publication
(10) Pub. No.: US 2024/0180417 A1
(43) Pub. Date: Jun. 6, 2024

(54) VISION TEST SYSTEM FOR SUBJECTIVE REFRACTION MEASUREMENT

(71) Applicant: EyeQue Inc., Newark, CA (US)

(72) Inventors: Noam Sapiens, Newark, CA (US); CATHERINE STARKS, Newark, CA (US); ANTHONY BAILEY, JR., Newark, CA (US); PATRICK O’NEAL, Newark, CA (US); JESSICA HSEUH, Newark, CA (US); John Serri, Newark, CA (US)

(21) Appl. No.: 18/386,529

(22) Filed: Nov. 2, 2023

Related U.S. Application Data
(60) Provisional application No. 63/421,666, filed on Nov. 2, 2022.

(51) Int. Cl. A61B 3/028

(52) U.S. Cl. CPC A61B 3/028; A61B 2560/0443

(57) ABSTRACT

The disclosed inventions include an embodiment of a mechanism, wherein an implementation of spherical power is through the concept of gaussian reduction:

$${∅}_{total} = {∅}_{1} + {∅}_{2} - {∅}_{1} {∅}_{2} \frac{t}{n}$$

where Øₜₒₜₐₗ is the total power applied by the two-lens system, Ø₁ and Ø₂ are the powers of the respective lenses, t is the thickness of the space between the two lenses and n is the index of refraction of the space between the two lenses. As seen in FIG. 5, this can be done with a two-lens system to generate optical power. Other implementations of the device could provide spherical power by using Gaussian Reduction with two Fresnel lenses.

### CROSS-REFERENCE TO RELATED APPLICATIONS

The utility application claims priority from U.S. provisional patent application 63/421,666 filed on Nov. 2, 2022. This application is a continuation or continuation in part of the following patent applications, the contents of which are incorporated herein by reference.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever. Such copyrights may include the term “EyeQue”

### BACKGROUND OF THE INVENTION

Vision is arguably the most important of the senses. The human eye and its direct connection to the human brain is an extremely advanced optical system. Light from the environment goes through the eye optical train comprised of the cornea, the pupil, and the lens and focuses to create an image on the retina. As all optical systems, light propagation through the eye optics is subject to aberrations. The most common forms of aberrations in the eye are defocus and astigmatism. These low order aberrations are the cause of the most common refractive eye conditions myopia (nearsightedness) and hyperopia (farsightedness). Higher order aberrations are also present and can be described most conveniently by the Zernike polynomials. These usually have a lower effect on visual function. The eye, like any other organ in the human body, may suffer from various diseases and disorders, the most prominent today are: cataract, AMD, glaucoma, diabetic retinopathy, dry eye. Other conditions exist and should also be considered in the scope of this application.

Ophthalmic measurements are critical for eye health and proper vision. Those ophthalmic measurements could be sectioned into objective and subjective types. Objective types measurements give a metric of a physiological, physical (e.g. mechanical or optical), biological or functional without the need for input from the measured individual (patient, subject, user or consumer). Examples of objective tests include but are not limited to OCT (optical coherent tomography used to image a 3 dimensional and cross sections of the eye), scanning laser ophthalmoscope (SLO, used for spectral imaging of the retina), fundus image (used to present an image of the retina), auto-refractor (used for refraction measurement), keratometer (used for providing a profile of the cornea), tonometer (used to measure the IOP—intra ocular pressure). Subjective measurements give a metric with relation to the individual input. That is, they provide parameters that also take into consideration the brain functions, perception, and cognitive abilities of the individual. Examples of subjective tests include but are not limited to visual acuity test, contrast sensitivity test, phoropter refraction test, color vision test, visual field test, and the EyeQue PVT and Insight.

Today, both objective and subjective eye exams (measurements) are done by an ophthalmologist or an optometrist. The process usually involves the patient needing to schedule an appointment, wait for the appointment, travel to the appointment location (e.g. office or clinic), wait in line, perform multiple tests using various tools and potentially moving between different technicians and different eye doctors. The prolonged wait times both for the appointment as well as in line at the appointment location, along with the hassle of performing the tests with different professionals and the duration of those tests might seem daunting to many patients. Furthermore, the sheer effort associated with the process and even the requirement of remembering to start the process to begin with might deter patients from going through with it.

Moreover, currently about 2.5 billion people do not have access to eye and vision care at all. The cost of eye exams could be considered quite significant especially in some places in the world. This poses a hindrance to the availability of eye care in third world countries for example. The cost, time consumption and perceived hassle also makes it at times prohibitive to have repeated eye exams, especially at the desired frequency. Those might be necessary in special cases (for example after refractive surgery or cataract surgery where repeated measurements should be performed to track the progress of the patient's status over time and the success of the surgery. Additionally, even under normal circumstances, measurements at a doctor's office only represent a single point in time. The situation under which the measurements were made might not be optimal or do not fully represent the patient's characteristics. The patient might have been tired, stressed or agitated (a doctor's visit might be quite stressful in and of itself but could also being run from test to test and being posed with questions and options elevate the patient's level of stress) or was just in a bad mood. Even the state of mind of the doctor themselves might influence the way the measurement is performed. Beyond all that, the time of day and other environmental conditions (whether direct e.g. lighting conditions or indirect e.g. temperature) could also affect the measurement and provide incomplete or false information.

The availability of information (including specifically medical information) on the internet, the increased awareness of people for preventive medicine, and the emergence of tele-medicine leads to many taking control of their own health. Devices for screening, monitoring and tracking medical conditions are quite pervasive in today's world, for example blood pressure measurement devices, and blood sugar monitors. The technological advancements allow for people to be more independent in diagnosis, prevention and tracking of various health conditions. Furthermore, many prefer to perform these activities in the comfort of their homes without the need for appointments or other time-consuming activities. In case of an anomaly, they would call or email their physicians to consult for the appropriate course of action.

The advancement of technologies effectively makes computers with screens and cameras ubiquitous in the form of laptops, tablets and smartphones. Therefore, enabling many people to have a device already capable of computing displaying and recording information.

All this brings the need for a series of devices that will enable users to perform ophthalmic measurements at home, by themselves, in a timely and cost-effective manner. It should be clear that the quality of these measurements and their accuracy and precision should meet or exceed the standards of today's measurement methods.

This vision could be further enhanced by use of cloud-based data and analytics that enables complete access to the entire history of a patient exams, tests and measurements. Moreover, the use of artificial intelligence (AI) will enable diagnosis based on machine learning and big data. This could be done by means of data mining, neural network decision making and pattern detection and recognition, as some examples of the AI capabilities.

To summarize, the vision for eye care in the not so far future will look like: A complete solution for eye and vision care for consumers and doctors; Remote, self-administered battery of tests for both disease and functional; measurements are enabled by technology and devices, AI is used for analysis, tracking and reporting. Enhanced by big data correlations and insights.

In simple terms, as an example: A person sits on their couch at the comfort of their home, uses a device to do various measurements, that data is uploaded to an AI for analysis. The AI will let the person know the results and notify the doctor. The AI will initiate alerts for the person and doctor in necessary cases. The person will not need to get up unless a serious issue occurs (i.e. surgery). All other issues will be dealt with remotely (e.g. email/phone/video conference with the doctor, order glasses and have them delivered to the home, direct delivery of doctor prescribed medications).

Despite the apparent approach of “direct to consumer”, the methodologies could easily be implemented for a more enterprise like model. One example of such implementation will have a hierarchical structure in which an entity such as a hospital, association, or a medical insurance company provides the ability for the doctors to provide their patients with such devices and capabilities. The devices are all connected through the user accounts to the cloud and the measurements are streamed directly into the users' accounts (and potentially their medical records). Those accounts could be attached to one or more doctors and can also be transferred and shared.

### (1) Field of the Invention

Disclosed embodiments generally relate to the technical field of optometry and ophthalmology; the measurement of refractions for users to correct vision; to adjustable glasses and other types of lenses

### (2) Description of the Related Art

The known related art fails to anticipate or disclose the principles of the present invention.

In the related art, existing technologies, while established, are antiquated in their ways of evaluating refractive measurements. These technologies, such as those shown in the prior art of FIG. 1 require extensive training, use of professionals at every step, and are very large in size and weight, require significant office space, command a large cost, etc. As such, the current field of technologies are only feasible in office settings and to those with extensive capital to expend. Often this means that offices providing help can be unaffordable for individuals who are the most in need.

In the current art, process of obtaining an optometric prescription, patients must set up an appointment, wait a period of time, and spend hours with an optician. Acquiring refraction measurements is a cumbersome process for many, particularly individuals who are in remote areas and would rather stay home. Setting up an appointment can also be time consuming for some. Moreover, on rare occasions, the refraction results could be incorrect, requiring the patient to return for an adjustment to their prescription.

Currently, there is a large time delay in obtaining refraction measurements and receiving glasses. There is yet no means to provide “instant gratification” and the ability to see with improved vision as soon as a user is given their prescription. To shorten that processing time, there is a need for a more mobile, smaller, cost-effective apparatus to provide users with a refraction measurement.

Some of the present disclosures, depicted in FIG. 2 and elsewhere present a significant departure from the prior art due to smaller form factor and weight, a focus on usability a providing a self-administered solution, providing immediate visual feedback of the corrected refraction, doing so while connected to software which enables computations of data trends and greater understanding of the user, and does so at a much lower monetary cost to the end user.

Thus, there is a long felt need in the art for the disclosed embodiments.

### BRIEF SUMMARY OF THE INVENTION

The present invention overcomes shortfalls in the related art by presenting an unobvious and unique combination and configuration of methods and components which provide a subjective refraction measurement capability that is embodied within an apparatus which provides an end user “instant gratification” or instant results of a user's current corrective need, and the ability to continually refine and improve upon that evaluated need. Two possible implementations of said embodiments can be seen in FIG. 3 and elsewhere herein

The disclosed embodiments include a method to negate the person's refractive errors in their own eyes, providing equal and opposite refractive errors within a disclosed system, evaluate these errors, and provide the refraction errors as correction values to the user. An example of such can be seen in FIG. 4.

These and other objects and advantages will be made apparent when considering the following detailed specification when taken in conjunction with the drawings.

### BRIEF DESCRIPTION OF THE DRAWINGS

FIG. 1 Elevation view of prior art and a disclosed embodiment

FIG. 2 Block diagram of disclosed embodiments

FIG. 3 Perspective views of disclosed embodiments

FIG. 4 Block diagram of disclosed embodiments

FIG. 5 Three vision train systems

FIG. 6 Elevation view of three lens systems

FIG. 7 Plan view of filter wheel

FIG. 8 Sectional view of a Stokes lens pair with rotational diagrams

FIG. 9 Two vision train systems featuring a Badal Lens

FIG. 10 Four testing views from a disclosed embodiment

FIG. 11 Sectional and perspective view of a disclosed rack and pinion system

FIG. 12 Perspective and exploded view of a disclosed rack and pinion mechanism sometimes used to execute a disclosed mechanism

FIG. 13 Sectional view of a disclosed rack and pinion mechanism

FIG. 14 Sectional and exploded view of a disclosed lead screw motion mechanism components

FIG. 15 Sectional and perspective view of a disclosed cylindrical optical mechanism

FIG. 15 Exploded and perspective view of a disclosed cylindrical optical mechanism

FIG. 16 Sectional and exploded view of a cylindrical optical mechanism

FIG. 17 Perspective view of cylindrical optical mechanism components

FIG. 18 Sectional view of cylindrical optical mechanism components

FIG. 19 Perspective and exploded view of interface rotational dial components sometimes used in an axis optical module

FIG. 20 Sectional view of axial optical mechanism and cylinder optical mechanism components

FIG. 21 Plan view of rotational movements of disclosed embodiments

FIG. 22a Sectional view of a phone centering mechanism

FIG. 22b Perspective view of a phone centering mechanism

FIG. 23a Perspective view of a pedestal mechanism

FIG. 23b Section view of a pedestal mechanism

FIG. 24 Perspective view of a box holder mechanism

FIG. 25a Elevational view of disclosed embodiments

FIG. 25b Elevational view of a disclosed embodiments

FIG. 26 Sectional view of a rack and pinion system contained within an outer housing

FIG. 27 Perspective view of a hinged pupillary distance measurement system

FIG. 28 Perspective view of a pupillary distance measurement system

FIG. 29a Perspective view of a pupillary distance measurement systems

FIG. 29b Perspective view of a pupillary distance measurement systems

FIG. 30 Perspective view of a four bar linkage system

FIG. 31 Schematic view of an optical system

FIG. 32a Perspective view of central hinge system with modularity attachment mechanisms

FIG. 32b Flipped perspective view of central hinge system with modularity attachment mechanisms

FIG. 33 Sectional views of magnetic attachment systems

FIG. 34 Sectional views of two modules attached via a spring loaded

mechanism

FIG. 35 Perspective view of a spring loaded slide mechanism

FIG. 36a Sectional view of an interlocking axle mechanism

FIG. 36b Perspective view of an interlocking axle mechanism

FIG. 37 Plan view of disclosed embodiments, a user and eye chart

FIG. 38 Perspective view of eyecups

FIG. 39a Perspective view of facemask with eyecups

FIG. 39b Perspective view of facemask with eyecups

FIG. 40 Perspective view of a facemask coupled with eyeglass like stabilization arms with PD mechanism of device shown.

FIG. 41 Perspective view of monolithic facemask cushion

FIG. 42 Elevation block diagram of electronics signal and data flow through a proposed system.

FIG. 43 Elevation diagram of data creation, interpretation, and display to the user.

FIG. 44 Diagram of optical system providing Spherical and Cylindrical refraction correction with field of view of 15 deg.

FIG. 45 Diagram showing simulation of expected “real life” view of system in FIG. 44.

FIG. 46 Diagram of optical system providing Spherical and Cylindrical refraction correction with field of view of 30 deg.

FIG. 47 Diagram showing simulation of expected “real life” view of system in FIG. 46.

### REFERENCE NUMERALS IN THE DRAWINGS

100 light rays from visual object.
101 negative power refracted light.
102 no power refracted light.
103 positive power refracted light.
104 power corrected light after invention.
200 mechanical componentry.
201 internal spherical lens holder.
202 spherical adjustment knob.
203 small pinion gear.
204 spherical module structure.
205 spherical encoder conversion shaft.
206 circular worm gear.
207 spherical lens 1 holder.
208 spherical lens 2 holder.
209 threaded cylinder barrel.
210 spherical structural mount.
211 cylindrical mount.
212 cylindrical lens 2 holder.
213 cylindrical lens 1 holder.
214 cylindrical structure mount.
215 gripped knob.
216 slide carriage.
217 ring gear.
218 double bevel gear.
219 ring to pinion gear.
220 pinion to bevel gear.
221 cylindrical lens 2 holder.
222 cylindrical threaded barrel.
223 knurled cylinder slide structure.
224 cylindrical lens 1 holder.
225 large adjustment gear.
226 gear shaft.
227 small adjustment gear.
228 friction pad.
229 friction lever.
230 elevated view of a phoropter module.
231 elevated view of binocular outer housing.
232 elevated model of module holder.
233 binocular module half.
234 sliding binocular module half.
235 sliding binocular base.
236 linkage module mount left.
237 linkage module mount right.
238 binocular right half.
239 binocular left half.
240 central pivot rod.
241 magnetic module half 1.
242 magnetic module half 2.
243 quick release structure base.
244 quick release structure attachment.
245 slide lever module base.
246 slide lever attachment structure.
247 binocular facemask module left.
248 binocular facemask module right.
249 central pivot attachment location.
300 view of optical pair 1.
301 elevated view of optical pair 2.
302 diagram of deformable lens.
303 spherical lens 1.
304 spherical lens 2.
305 cylinder lens 1.
306 cylinder lens 2.
310 Stokes Pair.
320 Badal Lens.
330 pair of cylinder lenses.
331 second set of cylinder lenses.
340 pair of spherical lenses.
341 second pair of spherical lenses.
400 electronic componentry.
401 rotary encoder.
402 electronics board.
403 electrical slip ring base.
404 electrical slip ring rotor.
405 slip ring combined.
406 electrical motor.
407 smartphone like electronics.
408 wireless connection.
500 componentry/mechanism for holding display devices such as smartphones.
501 section view of spring loaded phone holder.
502 whole view of spring loaded phone holder.
503 whole view of pedestal phone holder.
504 backplate component.
505 pedestal component.
506 pedestal structural mount.
507 snap close box phone holder.
600 diagram of an Eyeball.
601 visual targets.
700 componentry & mechanism which help to control pupillary distance.
701 elevated view of a PD connection mechanism.
702 PD rack gear top.
703 PD rack gear bottom.
704 PD pinion gear.
705 PD adjustment knob.
706 Hinge PD adjustment.
707 central hinge rod.
708 slide adjustment knob.
709 slide adjustment break.
710 slide adjustment channel.
711 thin sliding member.
712 thin sliding base member.
713 facemask structural plate.
714 set screw locking nut.
715 adjustment gear rack.
716 linkage structure bar 1.
717 linkage structure bar 2.
718 linkage sliding bar.
719 linkage coupled rotating bar.
800 componentry & mechanism which involve system modularity and/or attachment.
801 elevated view of modularity attachment.
802 lip attachment feature.
803 circular snap groove attachment feature.
804 circular snap hook feature.
805 groove attachment feature.
806 interlocking central pivots.
807 imbedded magnet.
808 interlocking alignment pin.
809 button head.
810 button pivot rod.
811 button spring lever.
812 button spring.
813 slide lever.
814 lever spring.
815 twist interlocking axle male.
816 twist interlocking axle female.
900 two interface components for a user's face or eyes.
901 eyecup.
902 left eyepiece of facemask.
903 right eyepiece of facemask.
904 eyeglasses like frame.
905 monolithic foam facemask cover.
906 a user's face.

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portion of this application.

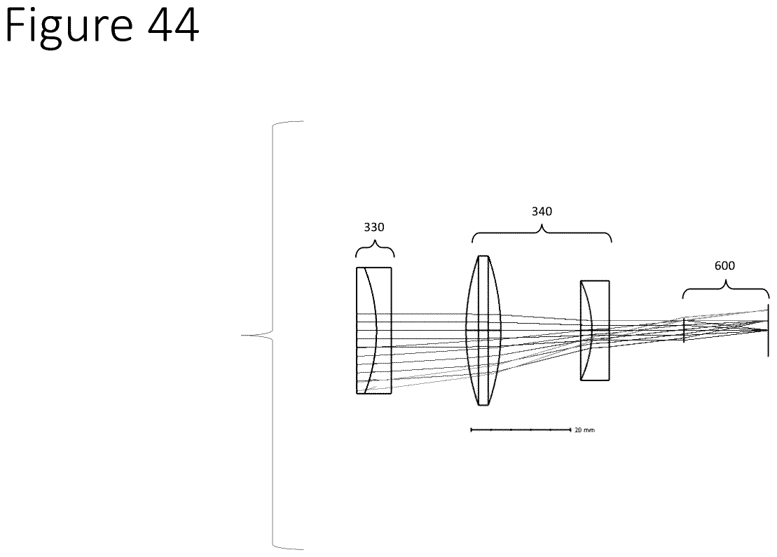

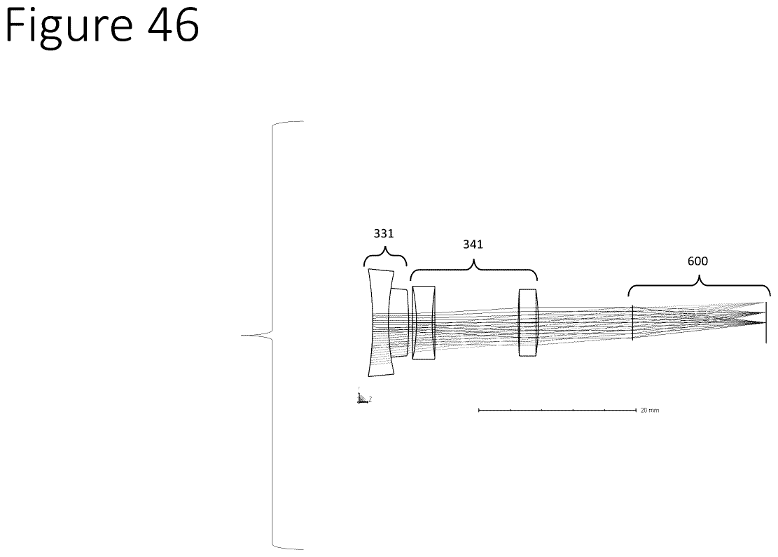

In a disclosed embodiment of the invention, the apparatus contains a mechanism for applying spherical power. In another disclosed embodiment of the invention, the apparatus contains a mechanism for applying astigmatism correction. In other possible disclosed embodiments of the invention, optical power can be created with any combination of spherical, and or astigmatic power correction. FIGS. 44 & 45 display an implementation of an optical system that has both spherical and cylindrical refraction correction capabilities with a field of view of 15 deg for the human eye. FIGS. 46 & 47 display another implementation of an optical system, which uses a different choice in optical lenses to accomplish spherical and cylindrical refraction correction with a field of view of 30 deg for the human eye.

In a disclosed embodiment of the invention, the apparatus will house a spherical optical module for performing refraction correction within a mechanism which provides spherical power opposite in sign to the spherical error of the user's eye through the movement and change of optical surfaces. The change in the optical surfaces can be mathematically understood to correlate to optical powers for refraction measurement.

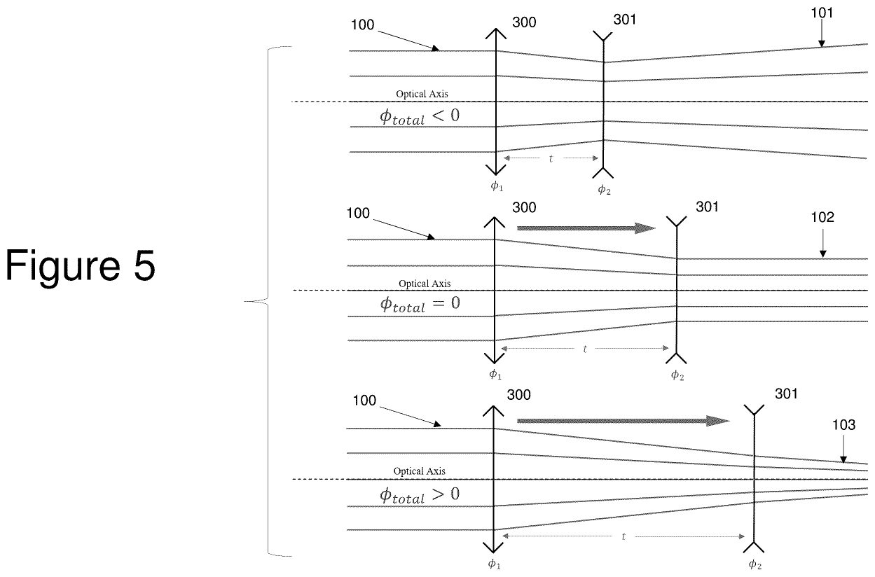

In this disclosed embodiment of the mechanism, an implementation of spherical power is through the concept of gaussian reduction:

$${∅}_{total} = {∅}_{1} + {∅}_{2} - {∅}_{1} {∅}_{2} \frac{t}{n}$$

where Øₜₒₜₐₗ is the total power applied by the two-lens system, Ø₁ and Ø₂ are the powers of the respective lenses, t is the thickness of the space between the two lenses and n is the index of refraction of the space between the two lenses. As seen in FIG. 5, this can be done with a two-lens system to generate optical power. Other implementations of the device could provide spherical power by using Gaussian Reduction with two Fresnel lenses.

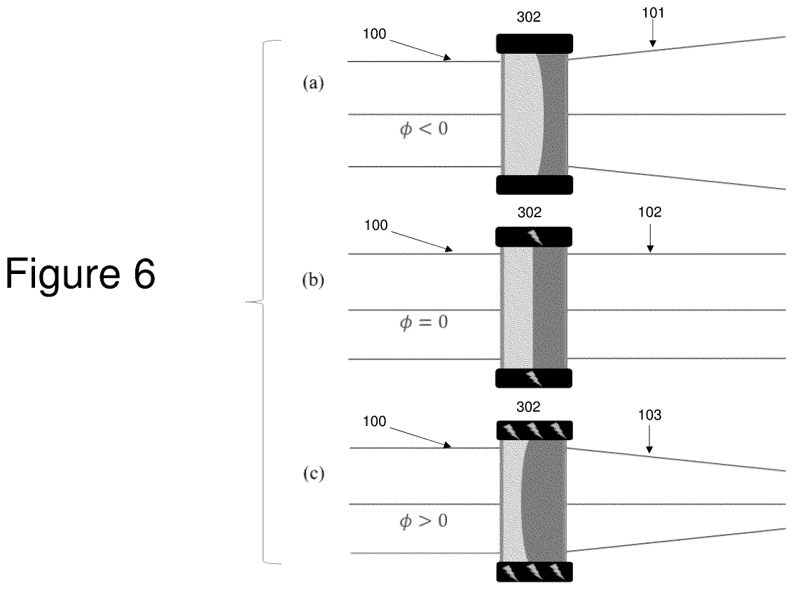

In this disclosed embodiment, an implementation of the mechanism can be a single deformable lens. FIG. 6 is a depiction of a deformable, or liquid lens. The lens contains a refracting liquid sack that changes its curvature as electrical current is applied, allowing the production of negative (A), 0 (B) or positive (C) optical power. This can be either a spherical or a cylindrical lens.

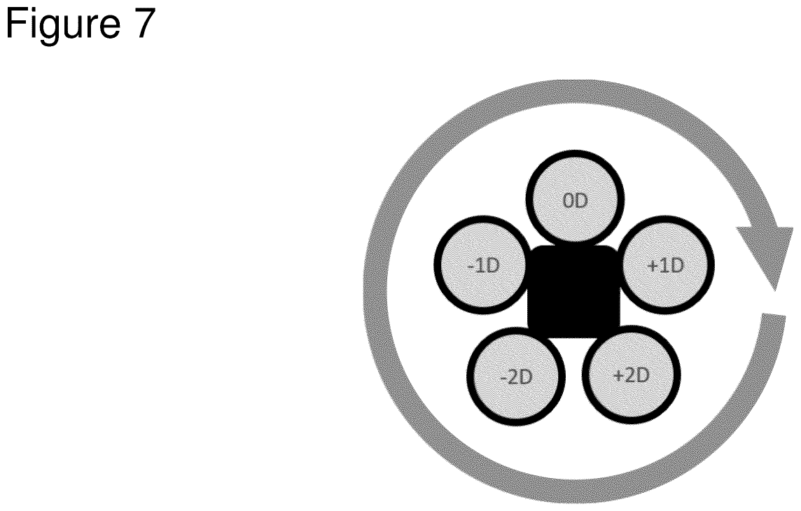

In this disclosed embodiment, an implementation of the mechanism can be a filter wheel containing lenses of various desired powers. FIG. 7 is a depiction of a filter wheel containing lenses of varying powers. The powers can be spherical or cylindrical. The lenses can be positioned into the optical path of the user and the user can indicate their preferred lens.

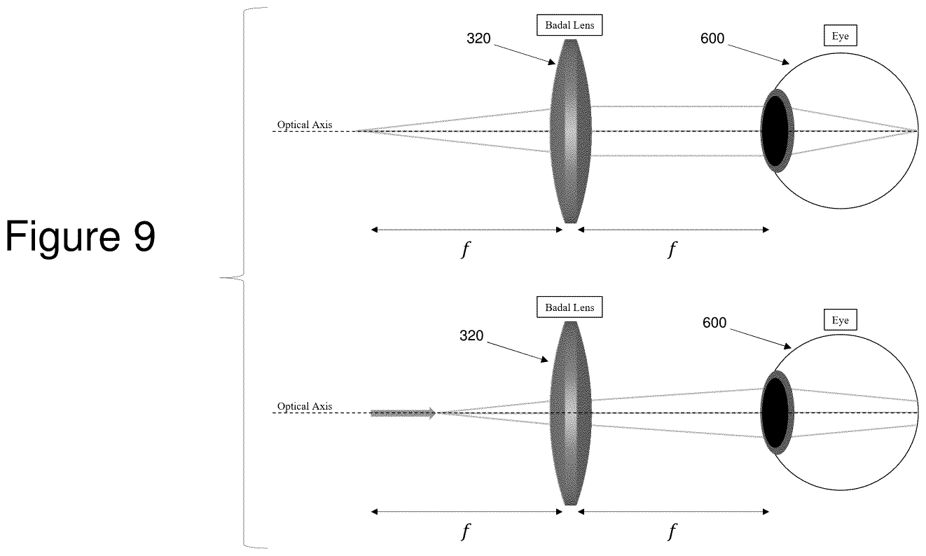

In this disclosed embodiment, an implementation of the mechanism can be a Badal lens. FIG. 9 depicts the use of a Badal lens. The lens is placed such that it is one focal length away from the eye, and a target is placed one focal length behind the lens. As the target is shifted forward or backward, the vergence of the light after the Badal lens changes. The target location can be shifted until the user views the target to be in focus, and the shift distance corresponds to the refractive error of the user. The Badal lens can be spherical or cylindrical.

In a disclosed embodiment of the invention, the invention uses a material between the optical components with an index of refraction n. In an implementation of the disclosed embodiment, the material between the lenses is air, with n=1. Other implementations could include water (n=1.33) between the lenses or other refractive mediums.

In a disclosed embodiment of the invention, the invention uses a two-lens system for applying spherical optical power to the system. This two-lens system will have capability to resolve a range of optical powers. In an implementation of the current disclosed embodiment, the system utilizes one positive and one negative spherical power lens to resolve both positive and negative powers. In other implementations of this embodiment the two spherical lenses can both be negative; this would provide only negative power to the system. In other implementations of this embodiment the two spherical lenses can both be positive, providing only positive power.

In a disclosed embodiment of the invention, the optical lenses are placed along the optical axis one in front of another. In an implementation of this embodiment, the lens furthest from the eye has a negative power and the lens closest to the eye with a positive power. Other implementations could have the negative lens closest to the eye and the positive lens furthest from the eye.

In a disclosed embodiment of the invention, the individual optical components within the system will have each their own spherical optical power. In an implementation of the embodiment, the invention utilizes spherical lenses that do not have equal and opposite powers; this causes magnification that does not equal 1 or −1. Other implementations of this embodiment could utilize spherical lenses with equal and opposite powers, yielding a magnification of −1, or spherical lenses with equal powers, yielding a magnification of 1.

In a disclosed embodiment of the invention, the optical components will have a mechanism to correct for off-axis aberrations. In an implementation of the embodiment, the mechanism comprises of one aspheric surface on the negative lens to correct off-axis aberrations with the remaining optical surfaces spherical. Other implementations could include no aspheric surfaces with no off-axis aberration correction or be several aspheric surfaces for off-axis aberration correction, or all lenses could be split into spherical doublets to correct for off-axis aberrations.

In a disclosed embodiment of the invention, the spherical optical module comprises of a mechanism that allows axial translation of the spherical optical components. This embodiment allows the invention to be able to accommodate for different user's spherical refractive errors. Possible implementations of this mechanism could be, but are not limited to, rotational to translational, translation to rotation, rotation to rotation, and more. These linear translation mechanics can occur through means such as, but not limited to, gearing, geartrains, rack and pinion, lead screw, frictional drive, human interface control, linear slide, and more not listed here. The positioning and motion can take the forms of linear, rotational, angular, complex motion, and other motion pathways.

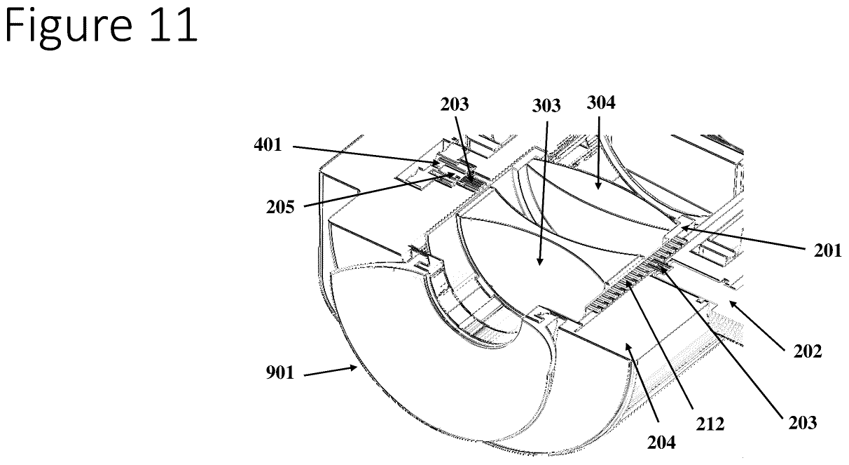

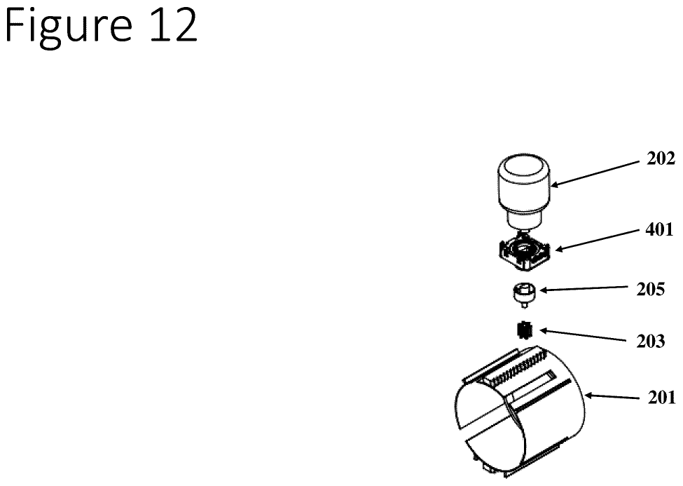

In the current disclosed embodiment, an implementation of an axial lens translation mechanism within the spherical optical module comprises of a rack & pinion like mechanism which slides a lens holder to and from a second lens. The pinion is rotated directly by the user and this action linearly translates the spherical lens and provides spherical power to the system. Different versions of this implementation can be seen in FIGS. 11, 12, & 13. These Fig.s depict views of a rack & pinion mechanisms along with internal details of the mechanisms used to provide motion axial translation to the lenses of the mechanism.

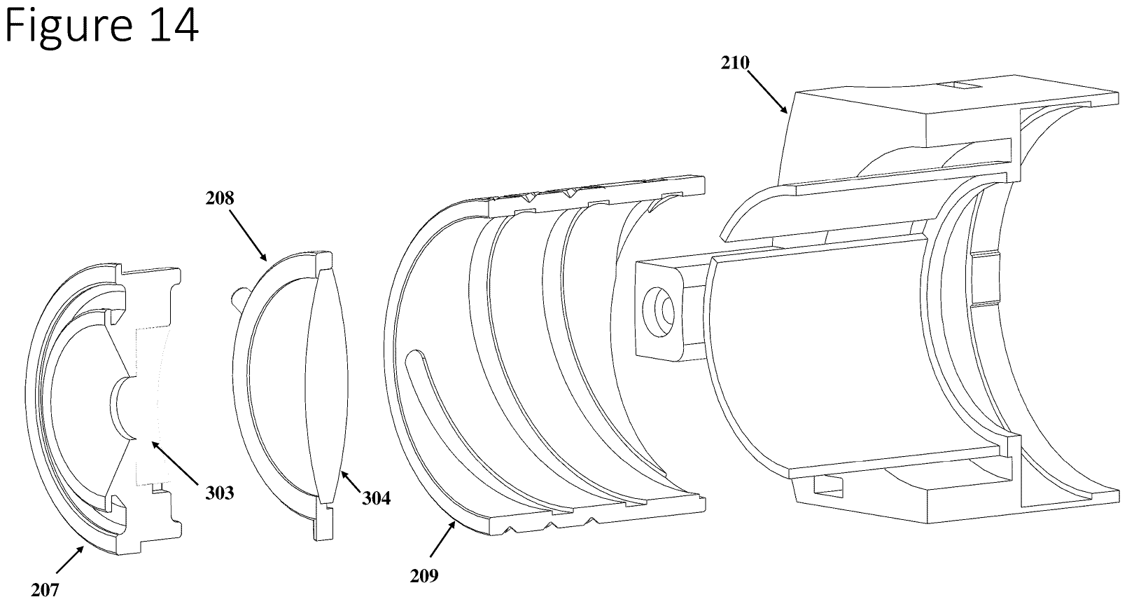

In the current disclosed embodiment, another implementation of an axial translation mechanism comprises of a lead screw mechanism which uses rotation motion of a body to create linear translation of a second body within the mechanism that contains a lens holder. By rotating one direction or another, the lens will move to and from the other lens, creating spherical power. This implementation and various components within it can be visualized in FIG. 14.

In a disclosed embodiment of the invention, the embodiment comprises of a cylinder optical module which provides astigmatic power opposite in sign and orthogonal to the cylindrical error of the user's eye. The change in the optical surfaces can be mathematically understood to correlate to cylinder optical powers for refraction measurement.

In the current disclosed embodiment of the cylinder optical module, cylindrical power is found by using the concept of Gaussian Reduction:

$${∅}_{total} = {∅}_{1} + {∅}_{2} - {∅}_{1} {∅}_{2} \frac{t}{n}$$

where Øₜₒₜₐₗ is the total power applied by the two-lens system, Ø₁ and Ø₂ are the powers of the respective lenses, t is the thickness of the space between the two lenses and n is the index of refraction of the space between the two lenses. An implementation of the embodiment can be seen in FIG. 5 where two cylindrical lens system is used to apply optical power.

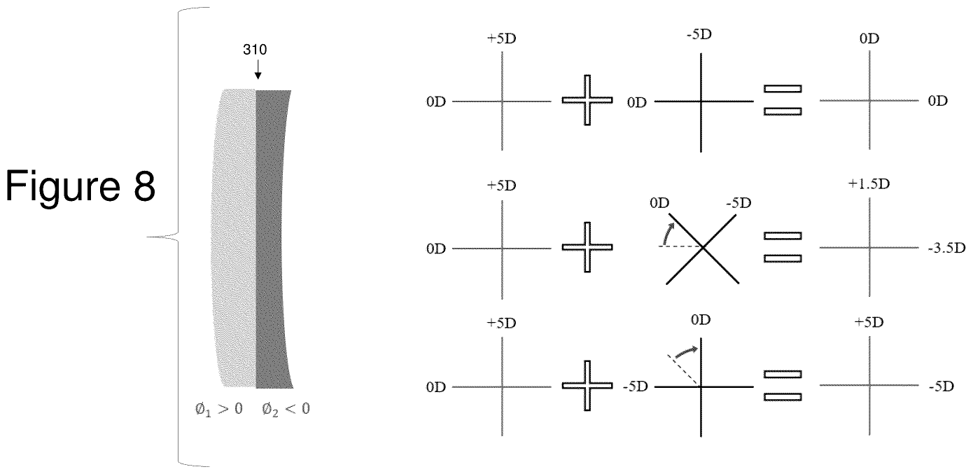

In the current disclosed embodiment, other implementations of applying cylindrical power can be using a Stokes pair of lenses utilizing a crossed pair of equal and opposite cylinder lenses. As seen in FIG. 8, a Stokes pair is two cylindrical lenses with equal and opposite powers are placed back-to-back. As the lenses are rotated with respect to each other, the resulting cylindrical power changes.

In the current disclosed embodiment, an implementation of the mechanism can be a single deformable lens. FIG. 6 is a depiction of a deformable, or liquid lens. The lens contains a refracting liquid sack that changes its curvature as electrical current is applied, allowing the production of negative (A), 0 (B) or positive (C) optical power. This can be either a spherical or a cylindrical lens.

In the current disclosed embodiment, an implementation of the mechanism can be a filter wheel containing lenses of various desired powers. FIG. 7 is a depiction of a filter wheel containing lenses of varying powers. The powers can be spherical or cylindrical. The lenses can be positioned into the optical path of the user and the user can indicate their preferred lens.

In the current disclosed embodiment, an implementation of the mechanism can be a Badal lens. FIG. 9 depicts the use of a Badal lens. The lens is placed such that it is one focal length away from the eye, and a target is placed one focal length behind the lens. As the target is shifted forward or backward, the vergence of the light after the Badal lens changes. The target location can be shifted until the user views the target to be in focus, and the shift distance corresponds to the refractive error of the user. The Badal lens can be spherical or cylindrical.

In a disclosed embodiment of the invention, the invention uses a material between the optical components with an index of refraction. In an implementation of the invention, the material between the lenses is air, with n=1. Other implementations of the embodiment could include water (n=1.33), or other fluids between the lenses.

In a disclosed embodiment of the invention, the invention uses a two-lens system for applying cylindrical optical power to the system. This two-lens system will have capability to resolve a range of optical powers. In an implementation of the current disclosed embodiment, the system utilizes one positive and one negative cylindrical power lens to resolve both positive and negative powers. In other implementations of this embodiment the two cylindrical lenses can both be negative; this would provide only negative power to the system. In other implementations of this embodiment the two cylindrical lenses can both be positive, providing only positive power.

In a disclosed embodiment of the invention, the optical lenses are placed along the optical axis one in front of another. In an implementation of this embodiment, the lens furthest from the eye has a negative power and the lens closest to the eye has a positive power. Other implementations could have the negative lens closest to the eye and the positive lens furthest from the eye.

In a disclosed embodiment of the invention, the individual optical components within the system will have each their own cylindrical optical power. An implementation of this embodiment utilizes cylindrical lenses that have equal and opposite powers; this causes magnification that equals −1. Other implementations could utilize cylindrical lenses with equal but not opposite powers, yielding a magnification of 1, or cylindrical lenses without equal and opposite powers, yielding a magnification that does not equal 1 or −1.

In a disclosed embodiment of the invention, the cylindrical optical components will have a mechanism to correct for off-axis aberrations. In an implementation of the embodiment, the invention has one acylindrical surface on the negative lens to correct off-axis aberrations with the remaining optical surfaces cylindrical. Other implementations could include no acylindrical surface with no off-axis aberration correction, or there could be several acylindrical surfaces for off-axis aberration correction, or all lenses could be split into cylindrical doublets to correct for off-axis aberrations.

In a disclosed embodiment of the invention, the optical components all share the same optical axis but may be arranged in differing groupings along that shared optical axis. An implementation of this embodiment has the cylindrical lenses furthest from the eye and the spherical lenses closest to the eye, but other implementations could have the spherical lenses furthest from the eye and the cylindrical lenses closest to the eye. Other implementations could have the optical elements may lie along a straight optical axis or the optical elements may lie along an axis that is folded by mirrors, prisms, and/or beam splitters.

In a disclosed embodiment of the invention, the cylinder optical module comprises of a mechanism which allows for axial translation of the cylinder lenses. Possible mechanisms to provide axial motion for these lenses could be, but are not limited to, rotational to translational, translation to rotation, rotation to rotation, and more. These linear translation mechanics can occur through means such as, but limited to, gearing, geartrains, rack and pinion, lead screw, frictional drive, human interface control, linear slide, and more not listed here. The positioning and motion can take the forms of linear, rotational, angular, complex motion, and other motion pathways.

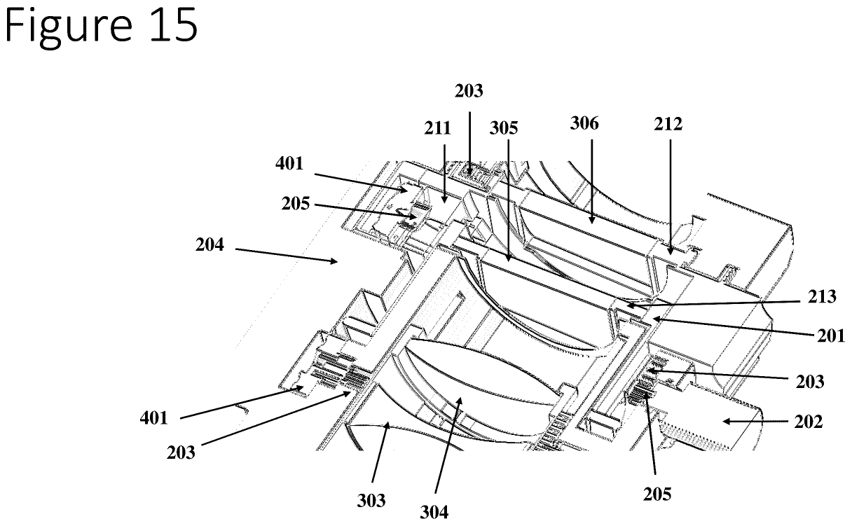

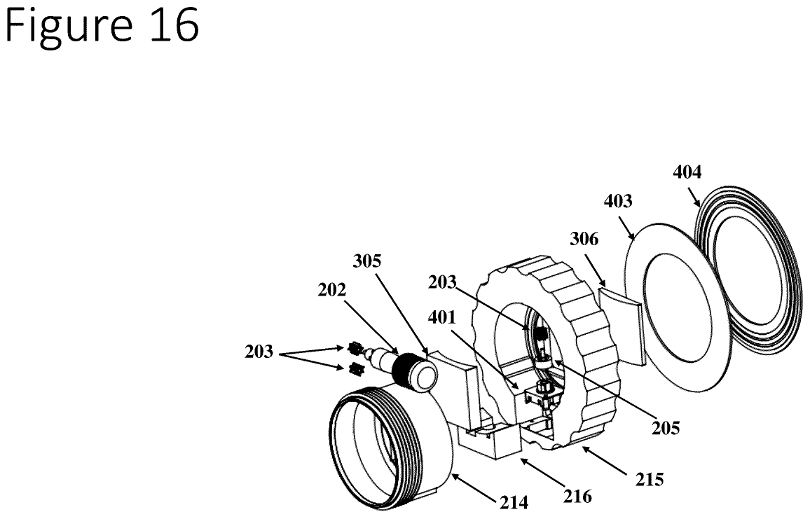

In the current disclosed embodiment of the invention, an implementation of an astigmatic optical mechanism comprises of rack & pinion like mechanism which slides a lens holder to and from a second lens. The pinion of this mechanism is rotated directly by the user and is the source of linear translation of one cylindrical lens to and from the other cylinder lens. Different versions of this implementation can be seen in FIGS. 15, 16, & 17. These Fig.s depict views of a rack & pinion mechanisms along with internal details of the mechanisms used to provide motion axial translation to the lenses of the mechanism.

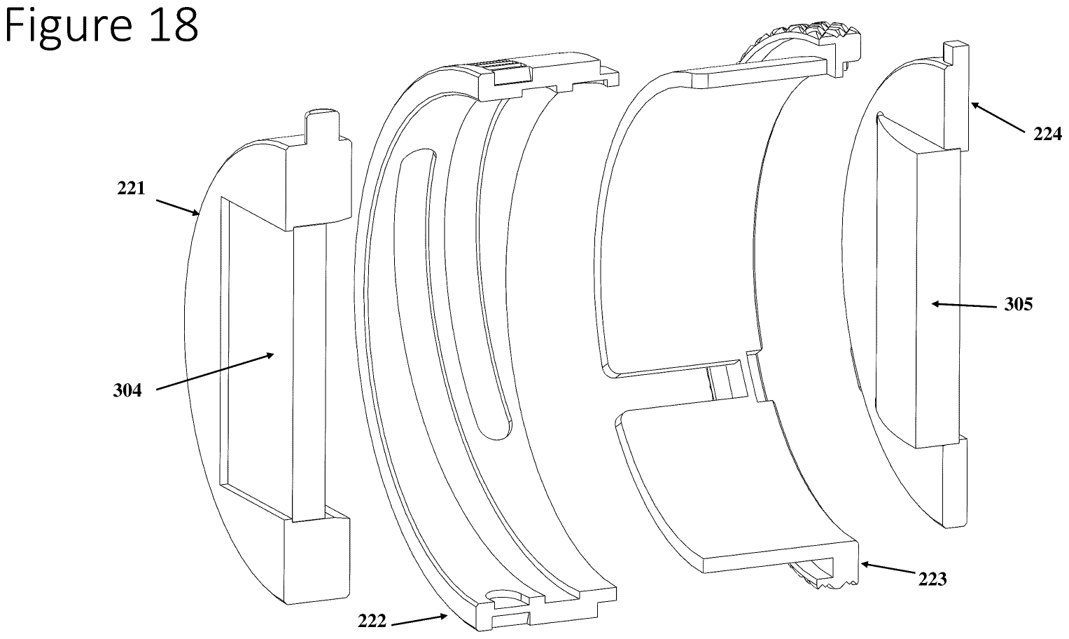

Another implementation of the current disclosed embodiment can be a lead screw mechanism that utilizes rotation motion of a body to create linear translation of a second body within the mechanism. The linearly translating body contains a lens holder and moves that cylindrical lens to and from the other lens within the system creating cylindrical power. This implementation and various components within it can be seen in FIG. 18.

In a disclosed embodiment of the invention, a astigmatic optical mechanism allows a user to correct for their astigmatism error. This astigmatism correction is done through rotational adjustment of the cylinder lenses within the astigmatic Optical Mechanism about the optical axis. Possible mechanisms to provide rotational motion for these lenses could be, but are not limited to, rotational to translational, translation to rotation, rotation to rotation, and more. These rotational motion mechanics can occur through means such as, but limited to, gearing, geartrains, rack and pinion, lead screw, frictional drive, human interface control, linear slide, and more not listed here. The positioning and motion can take the forms of linear, rotational, angular, complex motion, and other motion pathways.

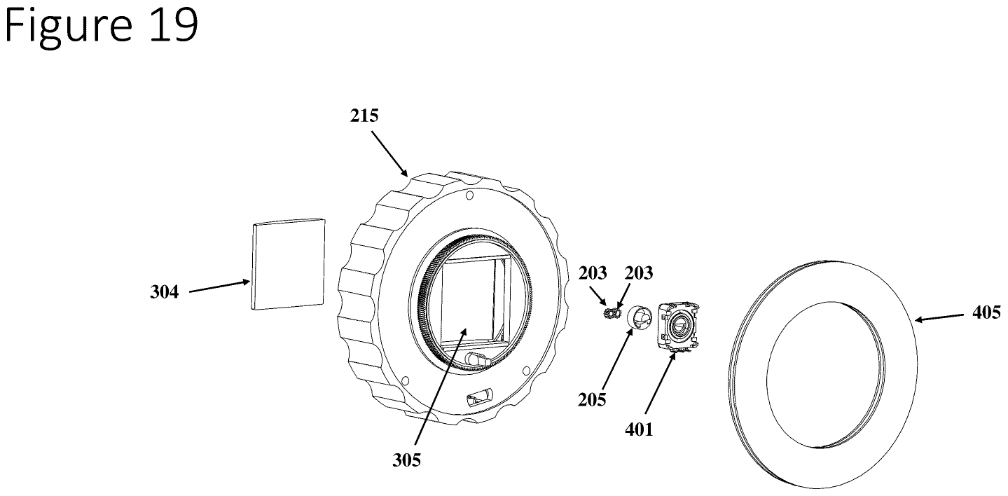

In the current disclosed embodiment of the invention, an implementation of a rotational mechanism for the cylindrical lenses could be housing the Cylinder Optical module within a rotational bearing surface and then utilizing a mechanism to rotate the Cylinder Optical Mechanism as a whole. This allows rotation without interference with translation. Two different versions of this implementation can be seen in FIGS. 19 & 20. Other implementations could be having the axial rotation control of the cylinder lenses within the cylinder optical module itself.

In a disclosed embodiment of the invention, the invention utilizes a known axial offset applied by the cylinder refractive optics to determine the user's axial correction need. An implementation of this embodiment can utilize the application of positive cylindrical power orthogonal to the cylinder axis of the user's eye. Other implementations could apply negative cylinder power along the cylinder axis of the user's eye by designing the cylinder lenses to apply negative power instead of positive.

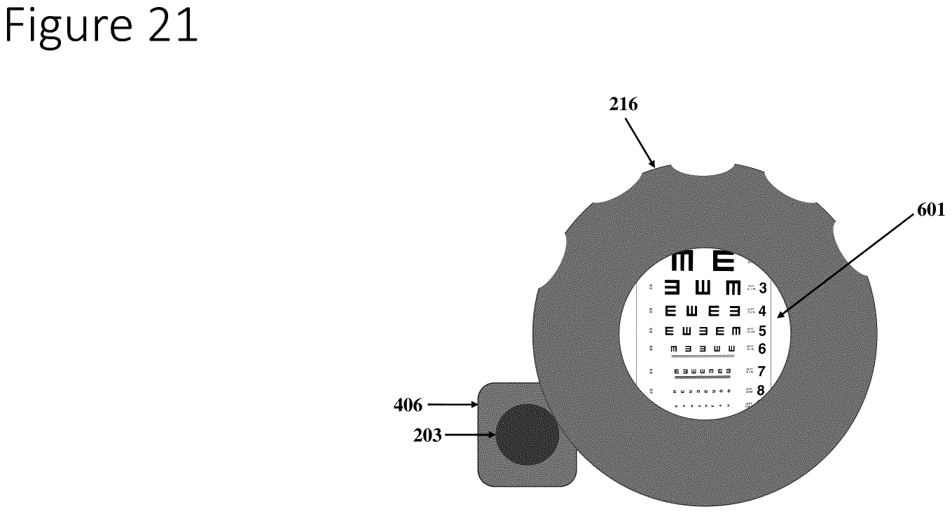

In a disclosed embodiment of the invention, the invention contains a means for producing motion in the optomechanical components such that the user can adjust the optical components until the user can see a clear image through the optics. An implementation of this embodiment can be human powered motion involving the change in position of wheels, knobs, levers, and more. Other implementations can be powered means of motion, such as motors, electromagnetics, piezo electrics, hydraulics, pneumatics, and other means of powered motion. FIG. 21 depicts aspects of both implementations mentioned, with motorized connections as well as human interface grooves.

In a disclosed embodiment of the invention, the invention requires that a user observes a visual object through the optical path. In an implementation of this embodiment, the user views a target at a fixed distance away to account for visual acuity and optical magnification. An example of the target in the distance is an ETDRS chart placed approximately 34 ft (10 meters) away from the viewer so that the lines in the letters on the 20/20 line of the chart subtend 1 arcmin on the user's retina (accounting for the current implementation's magnification factor). Other implementations of this embodiment can include a spokes wheel target, a Snellen chart, a general image, or a natural view where the viewer simply focuses on a distant scene in the world.

Other implementations of this disclosed embodiment could include a digital screen or a smartphone at the opposite end of the optical path from the user's eye. The screen or smartphone would display a visual with line widths such that they subtend 1 arcmin on the user's eye. This would keep the distance from the target to the user constant and the FOV can be greatly reduced. A de-magnifying device, such as the EyeQue Insight can be used in conjunction with either a screen or smartphone to project a target from a screen or smartphone onto the user's retina through the invention.

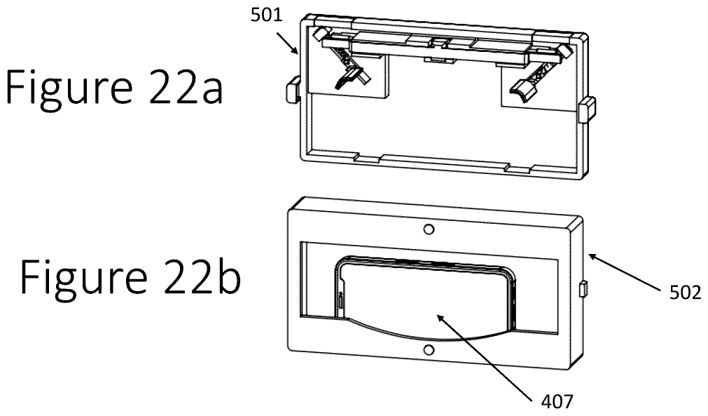

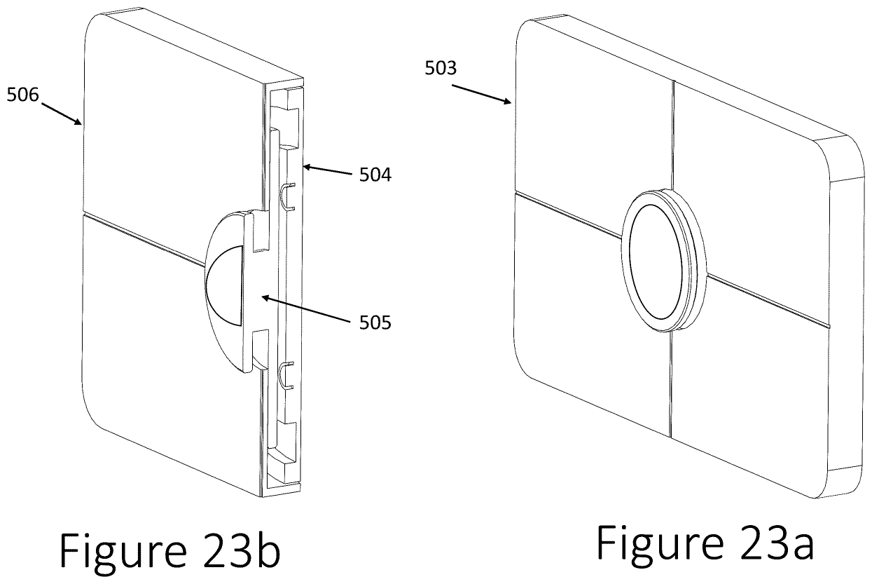

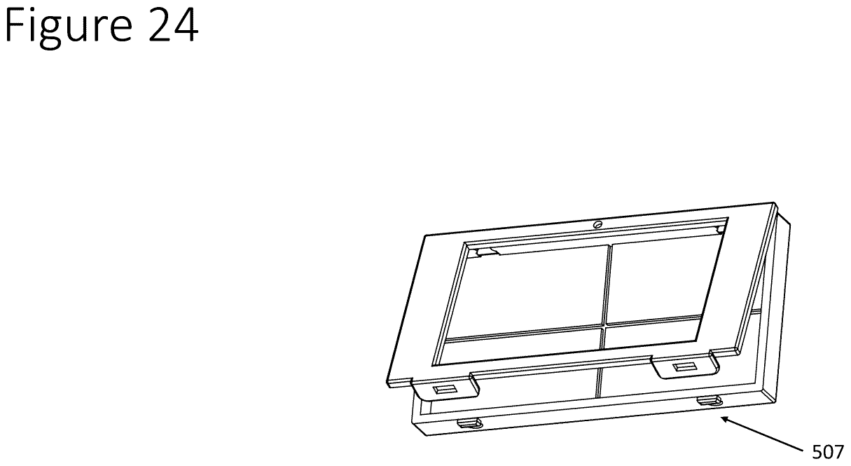

In a disclosed embodiment of the invention, visual information is displayed through a smartphone at the opposite end of the optical path from the user's eye. Utilizing a smartphone also requires that the invention has a mechanism for positioning the smartphone along the optical axis of the lenses. Implementations of this mechanism could look like a spring-loaded gripping mechanism which, at the behest of the user, applies an equal clamping force to the smartphone both gripping it and centering the smartphone in place. This can be seen in FIG. 22a, with the closed version in FIG. 22b. Another implementation could be a user adjustable pedestal that a smartphone is placed upon, as seen in FIGS. 23a & 23b, which grips the smartphone and, at the behest of the user, the pedestal can be moved with the phone to align the phone screen to alignment features within the apparatus. Another implementation could be a simple box with snap lid mechanism as seen in FIG. 24. The user places the phone within a box with alignment features, at which the phone is held in place when the lid to the box is closed.

In a disclosed embodiment of the invention, the invention uses a specific FoV designed to meet the user needs for observing the visual object through the optical path. In the current implementation of the invention, the FoV is designed for a 30° Full Field of View (FFOV). This is considered the symbol recognition field of view. Other implementations of the apparatus could be designed for a larger FOV to allow the user a more complete view of the world, however off-axis aberrations would have to be dealt with. Other implementations could be design for a smaller FOV, but the user's view of the world would be limited.

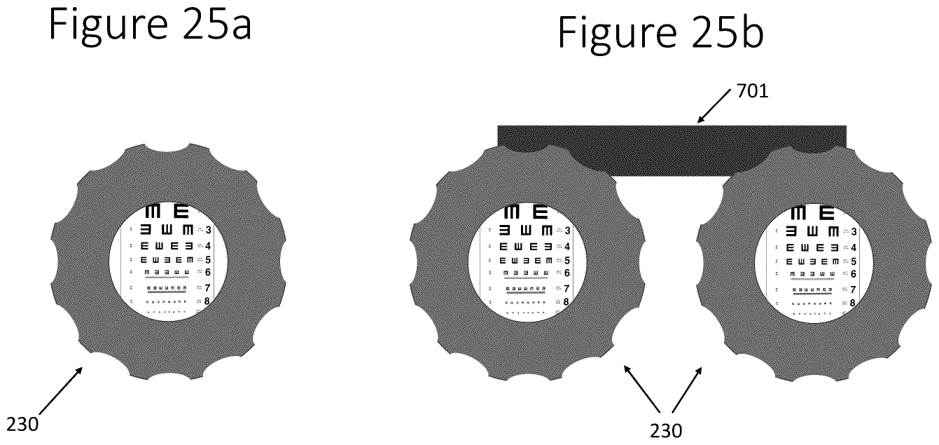

In a disclosed embodiment of the invention, the invention comprises of several optical pathways needed to appropriately provide a user their optical refractive correction. In an implementation of this embodiment, the invention is monocular, meaning that there is only one optical axis as seen in FIG. 25a. Other implementations of the device could be binocular to mitigate accommodation, as seen in FIG. 25b. Binocularity of the apparatus may also include the ability to allow users to adjust their pupillary distance (PD). A binocular device could allow the user to find the refraction correction values of both eyes at nearly the same time, effectively working as adjustable, tunable eyeglasses. Other implementations could have as many optical channels as needed to accomplish the task of providing the user with their optical refraction correction.

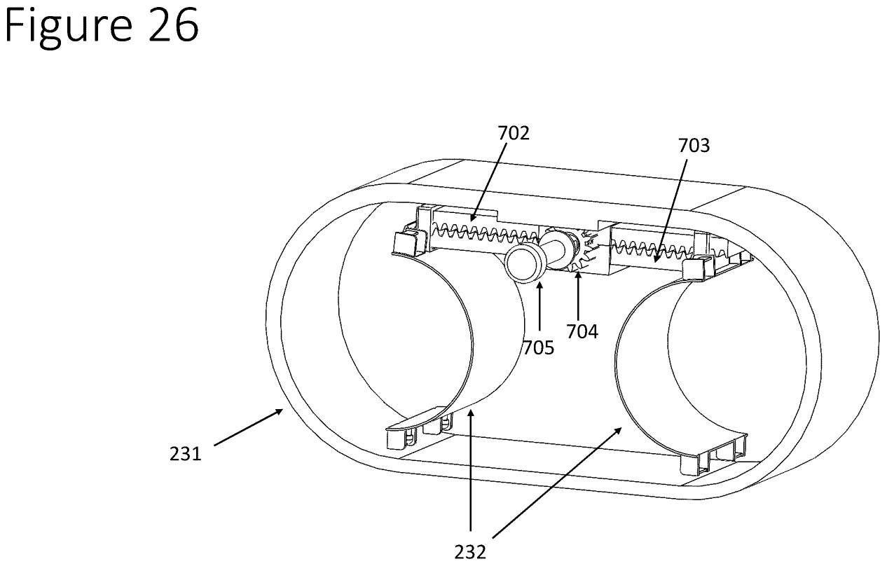

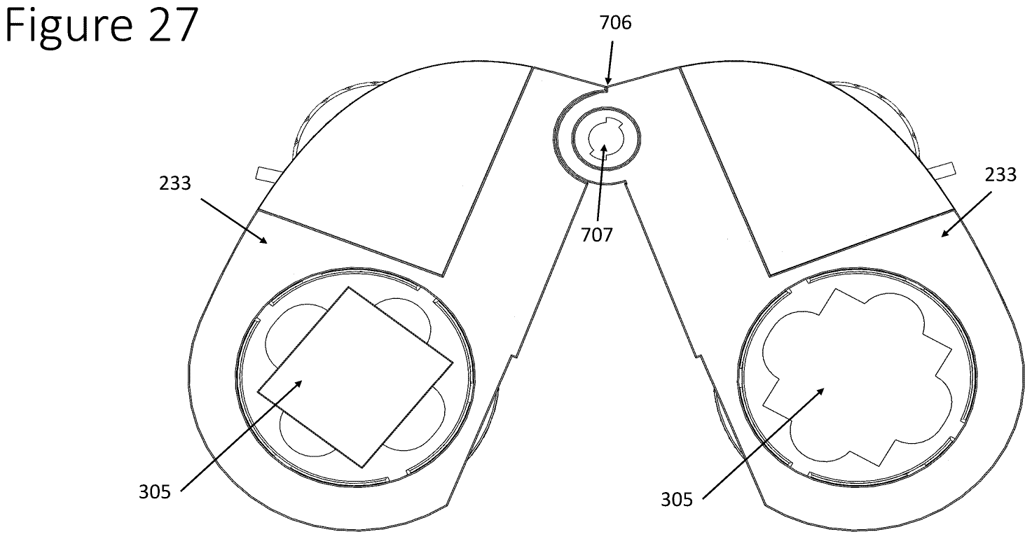

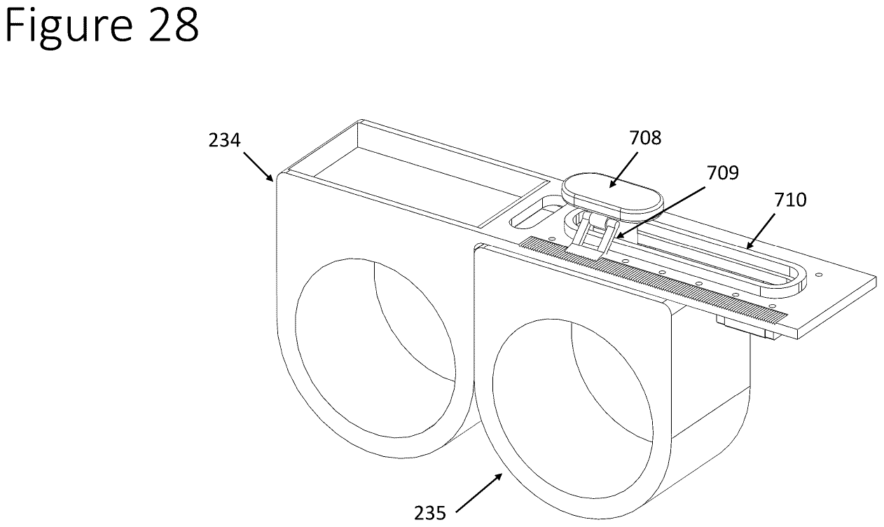

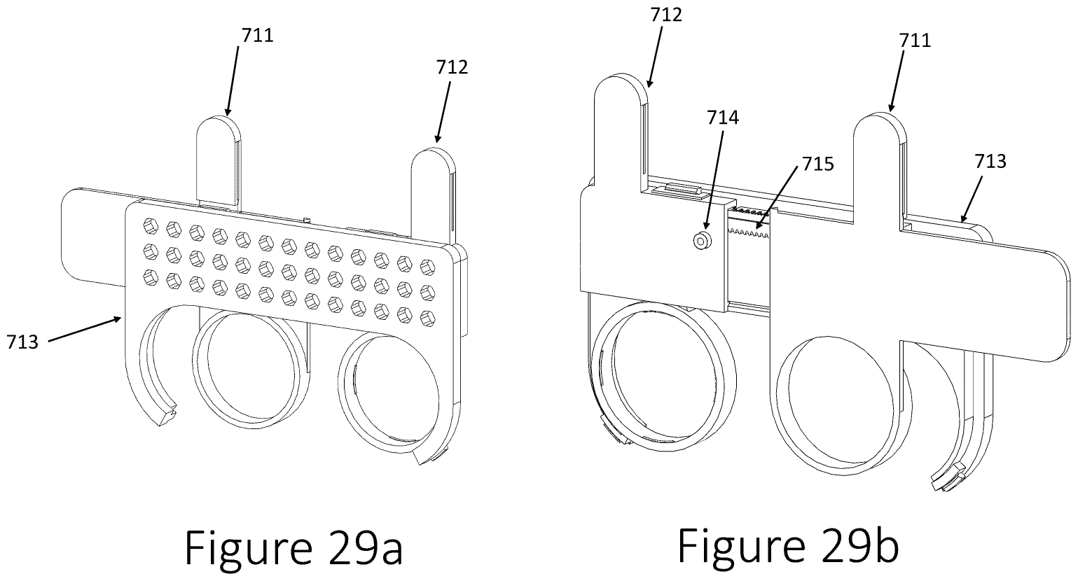

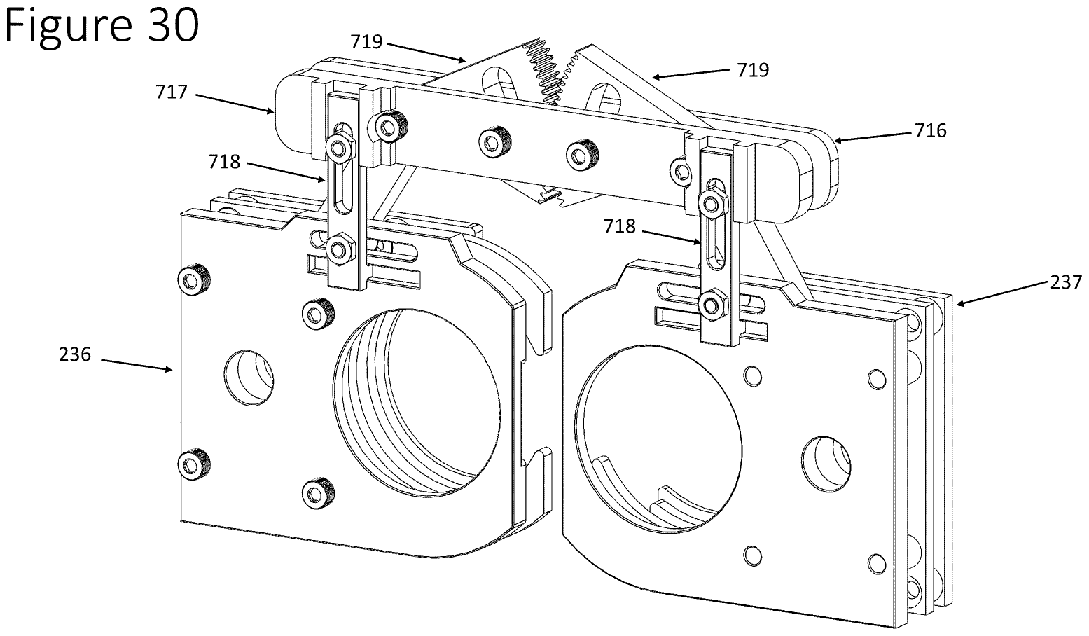

In a disclosed embodiment of the invention, the invention is a binocular device with two optical channels, one for each eye. To enable this embodiment to accommodate a range of users, a mechanism would be needed to account for the possible range of pupillary distances for each user. An implementation of a mechanism to enable this binocular embodiment could be rack & pinion system that has a rack attached to each optical channel. As seen in FIG. 26, the pinion is directly controlled by the user which, when rotated, adjusts the pupillary distance of the apparatus through the racks attached to each channel. Another implementation can be seen in FIG. 27 with a hinged central pivot mechanism which the optical channels rotate about a central pivot point until the user achieves the desired PD. Another implementation can a linear slide mechanism, as seen in FIG. 28, in which one channel moves to and from another channel along a rail fixed to the other channel. FIGS. 29a & 29b shows a similar implementation using a more flattened sliding rail design. Another implementation of this mechanism can be a self-leveling linkage mechanism which allows two optical channels rotate about a pivot point while maintaining the Axis orientation to the user. This implementation can be seen in FIG. 30.

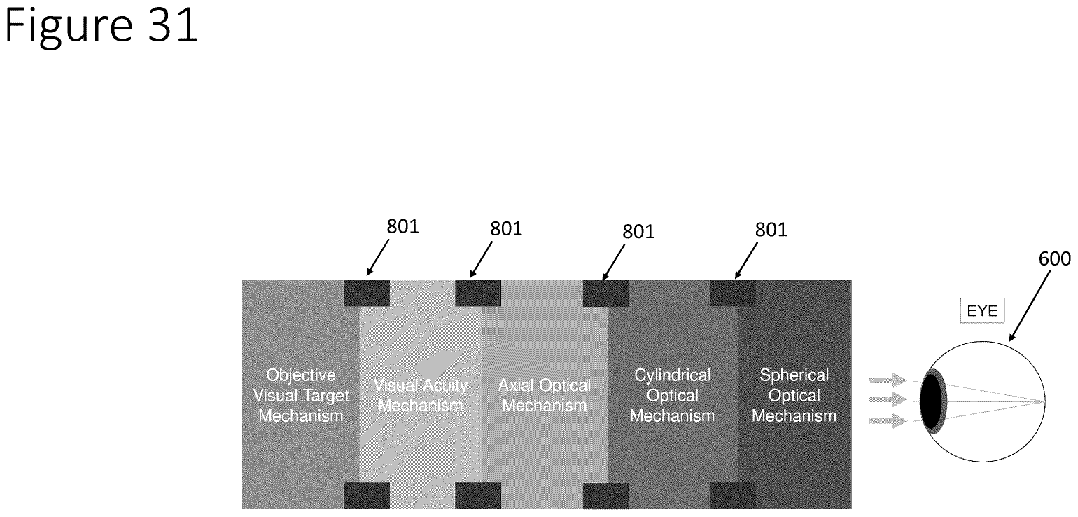

In a disclosed embodiment of the invention, the invention will be modular in design, allowing for varying combinations of features that can be custom tailored to the need of the user. A modular design could allow the user to measure their spherical and/or cylindrical values separately, or attach another optical component onto the apparatus, including but not limited to, phone holders, virtual reality displays, or color filters. An implementation of this embodiment can be seen in FIG. 31, displaying an assembled modular invention. In this implementation any number of modules may be present, in which the optical surfaces to measure spherical power, cylindrical, and/or axial powers are in any number of separate modules. These modules can be combined to perform different functionalities.

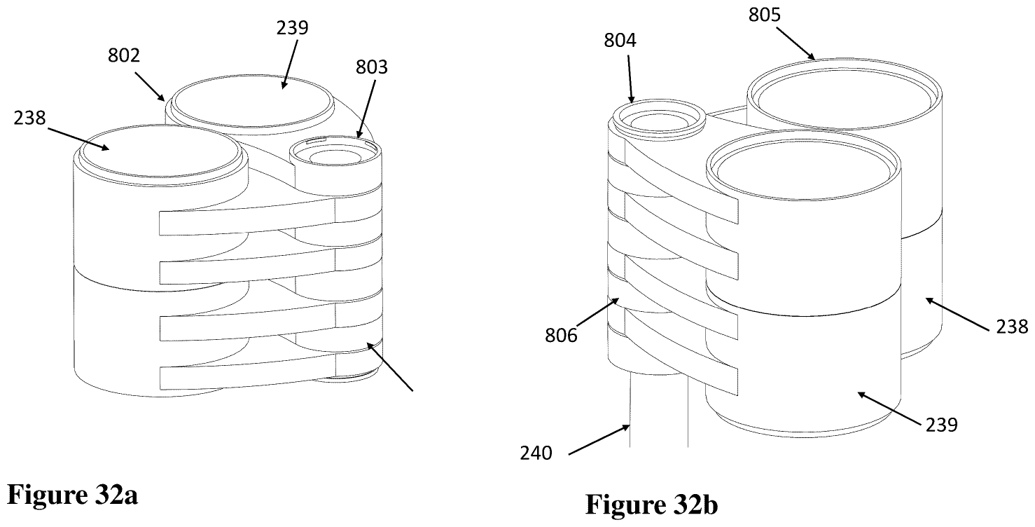

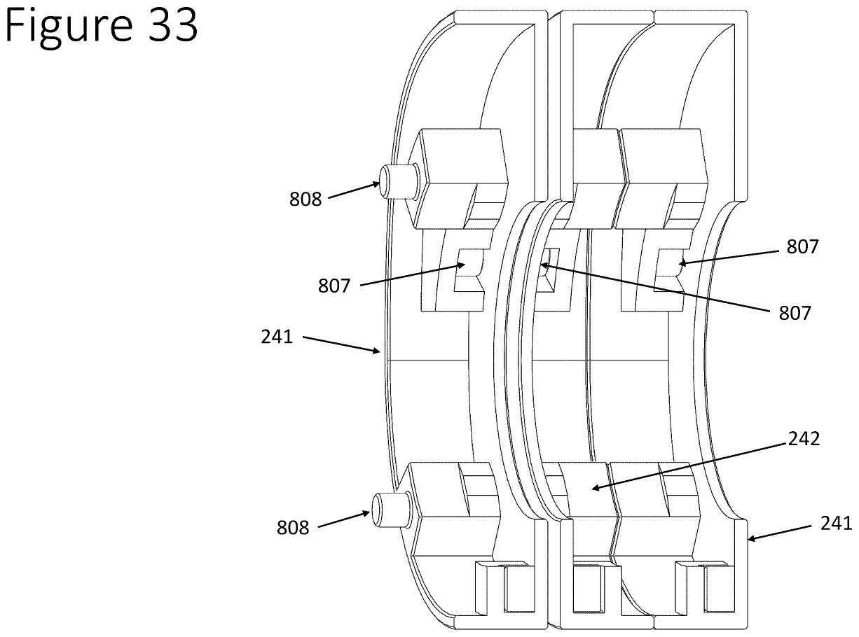

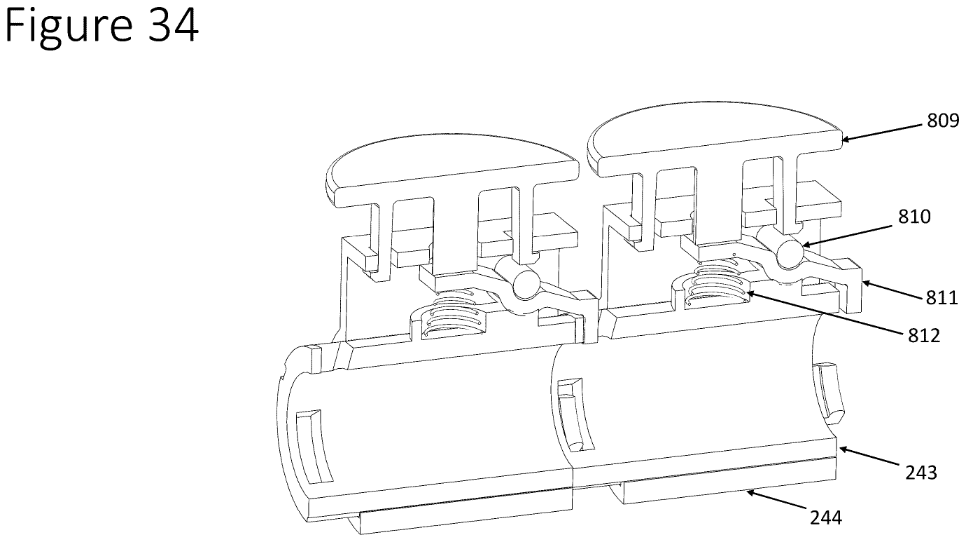

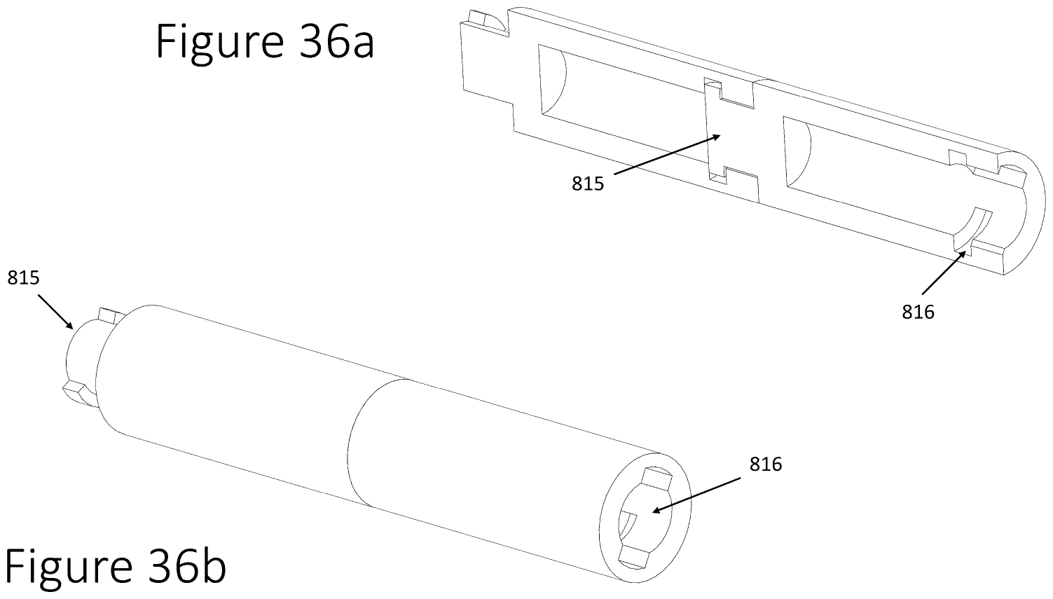

In a disclosed embodiment of the invention, modularity is enabled through physical attachment mechanism which allow for these modules to interface with one another. Several implementations of this physical modularity can be seen in FIGS. 32a & 32b, such examples are Lip and Groove interlocking features, or a Central Pivot Pin alignment feature where each module would attach to and share a central shaft axis used for mounting and stability, or Snap Fit attachment features where each module contains corresponding snap fit hooks and grooves for each module with likely other alignment features to assure snaping. FIG. 33 shows other possible implementations such as magnetic attachment features where opposite polarity magnets are utilized, likely in combination with other alignment features, to attach the modules together. FIGS. 34 & 35 show additional implementations such as a spring loaded or stored energy attachment mechanisms where a hook, lever, or latch is engaged upon the release of a button, lock, or other fixture which then attaches one module to another. FIGS. 36a & 36b showcases on other implementation which involves an interlocking axle mechanism where pins, axles, or other central structural members are attached and interlocked to form a rigid body.

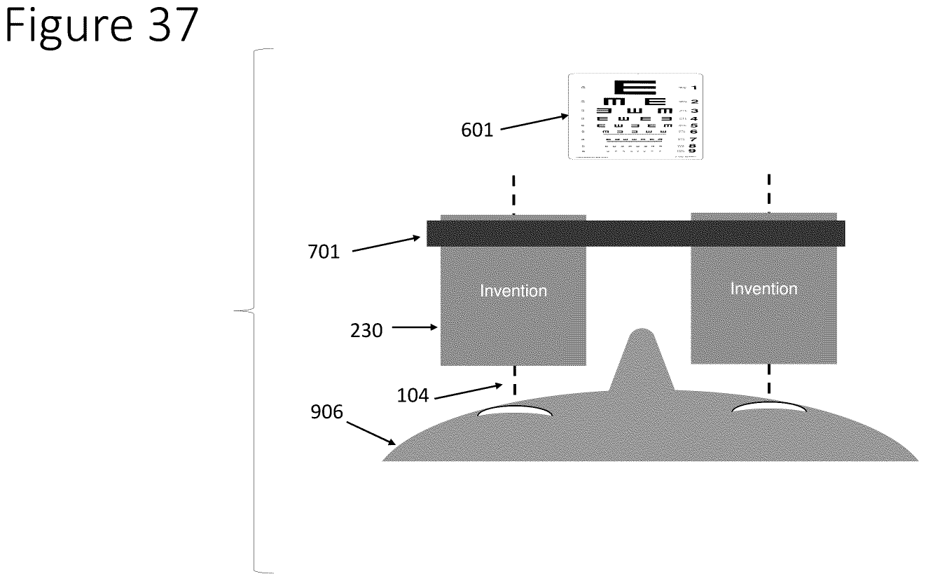

In a disclosed embodiment of the invention, the invention requires alignment of each of the device's optical channels to the end user's eye. An implementation of this embodiment is to require the user to align themselves to the apparatus. Other implementations of alignment could be device assisted/self-administered, or this alignment can be automated through the apparatus. Another implementation could be the invention requires a technician or trained individual to assist the user. These implementations can occur through any number of mechanisms such as mechanics, optics, or electronics to assist the user. FIG. 37 showcases an example of a dual channel system being aligned to a user's eyes via mechanism which may exist within the space of the invention, or through other means listed above.

In a disclosed embodiment of the invention, the invention will have an interface for a user that allows the user to perform the vision tests comfortably, easily, and safely with regards to contamination. In an implementation of this embodiment, the interface will align the user's eye to the optical path such that the first optical lens is the appropriate distance away from the user's eye, and the user can adjust the interface so that the center of their pupil follows the center of the optical path. This implementation also will utilize an interface with the user such that it can be decontaminated at the user's discretion, or be self-cleaning, to prevent transmission of unwanted particulates/biological matter.

In a disclosed embodiment of the apparatus, the invention will incorporate mechanisms to block unwanted light from entering the eyes of the user from outside the optical pathway. This excess light would inhibit accurate measurements of the user's refraction values. Implementations of these mechanisms can be, but are not limited to, FOV limiting within the apparatus, physical blockages of light around the user's face/head, having the tests be performed dark locations, and more not listed. Several mechanisms can incorporate implementations of a facemask, eyecup, or other face covering which may include attachments to allow the smart phoropter to be supported in a hands-free manner, such as using headbands, head straps, glasses arms, ear hooks, hat features or other options not listed here. These attachments may be adjustable to allow the device to fit a variety of people and they may be removable to allow the user to swap different attachments for improved comfort. To increase comfort and ease of usability for the user.

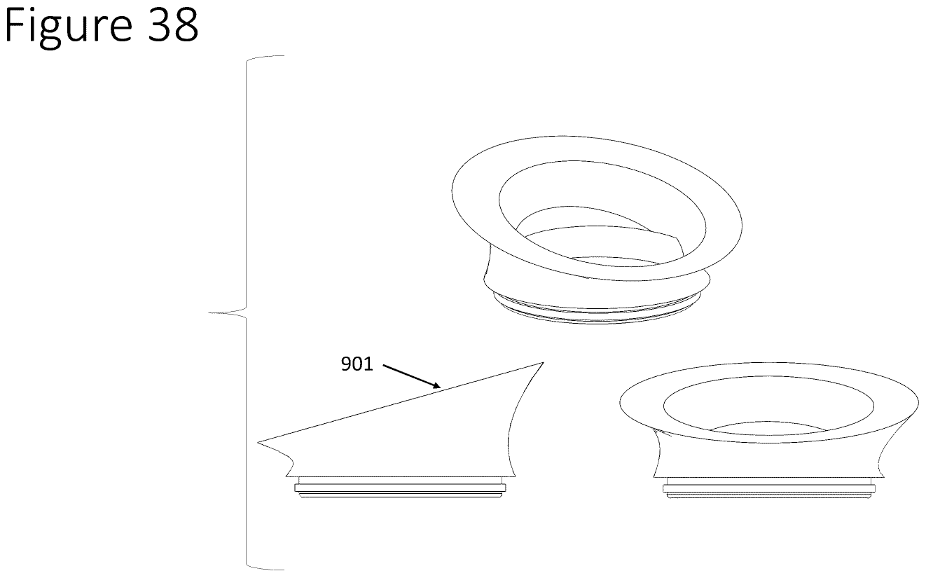

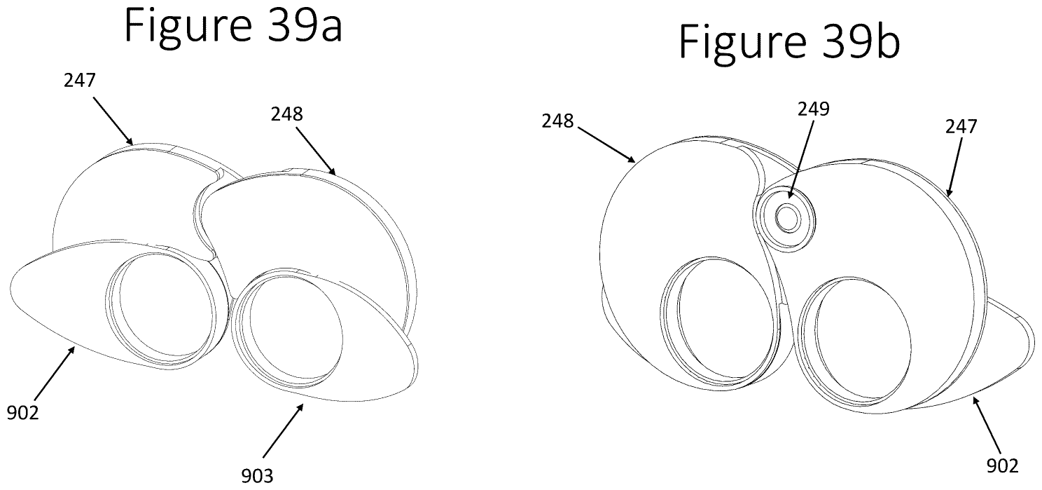

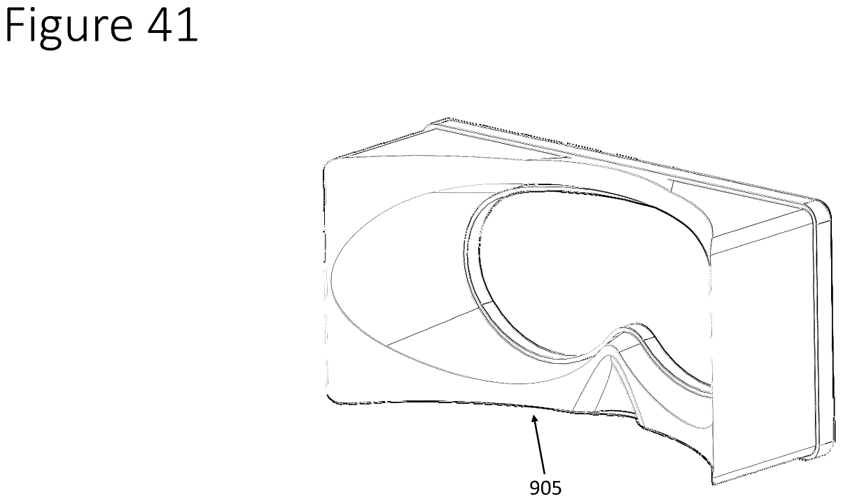

In implementations of the current disclosed embodiment, the stray light is blocked using eyecups which surround a user's eye and provide completely coverage which can be seen in FIG. 38. Other implementations of this could be a structural facemask which covers a large portion of the user's face that blocks external straight light while allowing a more supportive and comfortable rest for the user. This facemask may be implemented in a manner seen in FIGS. 39a, 39b, & 40 where the mechanism contains a PD mechanism to adjust for a user face and may provide a cushion. Another implementation could be a flexible facemask, seen in FIG. 41, where the facemask is a single static cushion piece that is designed to accommodate a wide swath of user head shapes and sizes while having imbedded into it a means for accommodating PD of the modules.

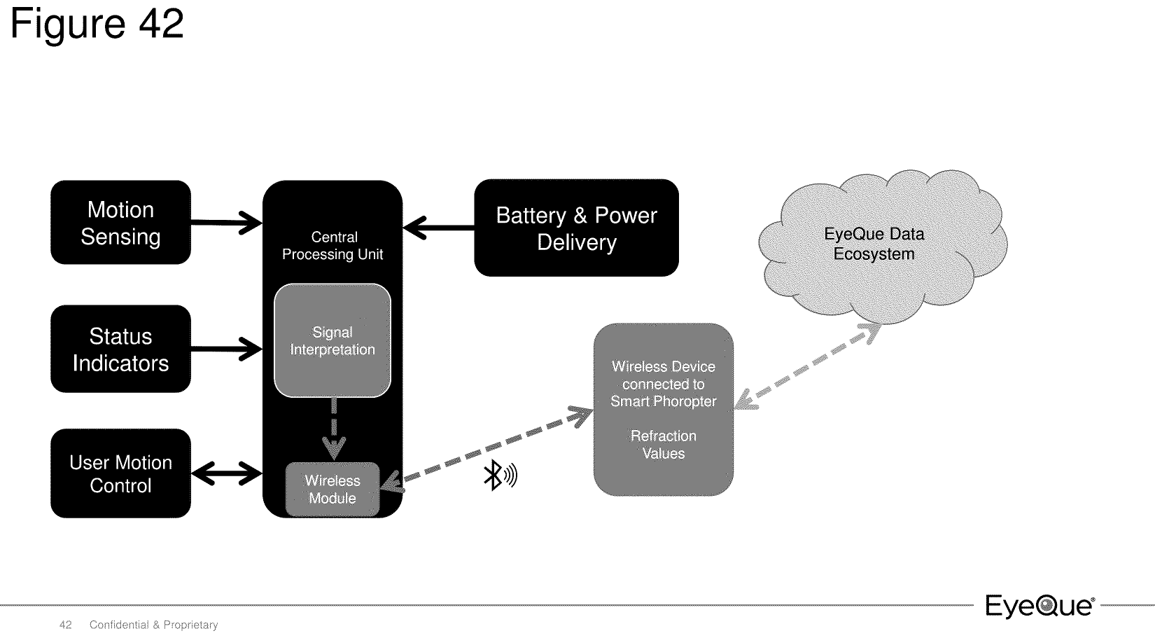

In a disclosed embodiment of the invention, there is a mechanism for sensing the active, real-time status of the invention to measure current optical refraction power for the user. This real-time status can involve, but not be limited too, change in optical element position or orientation, change in electronics state, change in software state, change in the user state, or change in connectivity of any connected devices. A diagram denoting a version of this interconnectivity can be seen in FIG. 42.

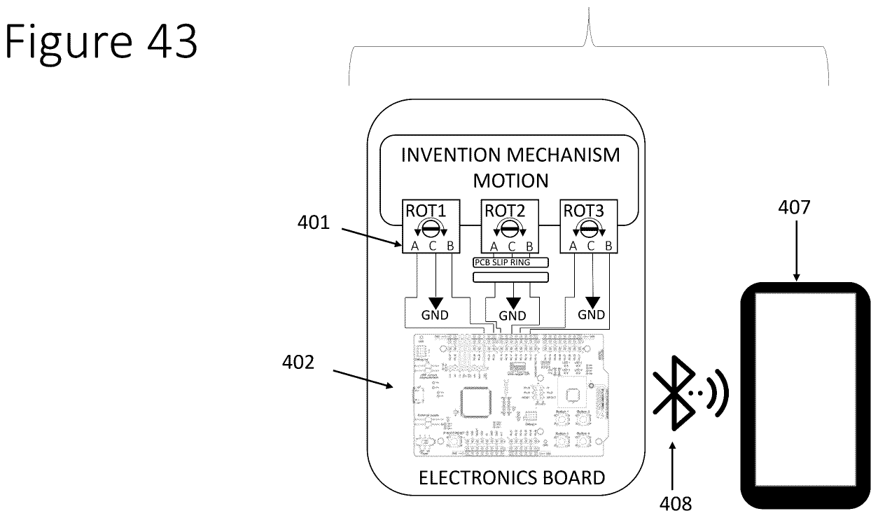

In a disclosed implementation of the current embodiment in FIG. 43, change of state is sensed through electromechanical means which detect the change of mechanical state and translate that to an electronic signal representing that change. The existing implementation uses rotary encoders attached to key features of the mechanical mechanism to sense change in optical state. Other implementations of sensing state change can be through linear encoders, limit switches, line of sight sensing, ultrasonic sensing, optical reflectance sensing, and/or interrupt sensing.

In a disclosed embodiment of the invention, the invention will contain electronics as needed to assist in performing refraction correction, proper functionality, which will need to electrically connect to one another regardless of the level of modularity of the device. An implementation of the embodiment, the electrical connectivity can be completed through such manners as spring loaded pin to pad contacts, or barrel to jack connections like in audio applications, and/or wireless protocols like Bluetooth or NFC.

In a disclosed embodiment of the invention, the invention contains a central processing unit which is utilized for the computation of sensory state data into user's refractive error. The sensor system sends electronic signal data to this processing unit which, through software, converts this data until refractive values for a user to understand. An implementation of this embodiment can be seen in FIG. 42 is a microprocessor board within the invention which coordinates all the data collected by the device, organizes it, and presents it to the user. Other implementations could be interfacing via wireless means to a smartphone, tablet, or computer for computation. Another implementation could be non-electronic means which involve direct readout of a user's values from mechanical conversion of spatial movements into an interpretable distance value on the invention itself.

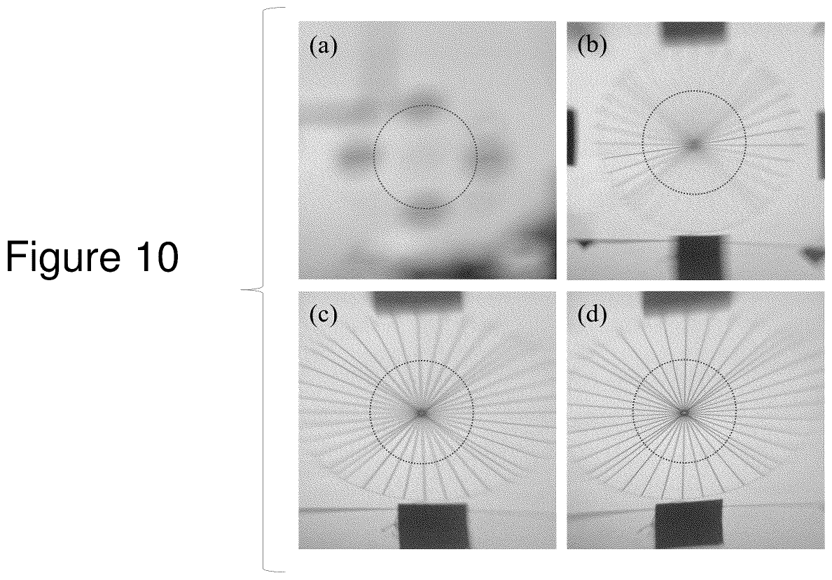

An embodiment of a method for assessing a user's refraction effort using the invention can be conducted through the procedure noted below. The steps in correspond to the images within FIG. 10.
(1) The user puts the apparatus up to their testing eye and looks through the apparatus at the visual object, which should appear blurry.
(2) In an implementation of this embodied procedure, the user first adjusts the Spherical Optical Module of the apparatus to add spherical power to the system. The user stops adjusting the Spherical Optical Module when lines that are spread along the user's astigmatic axis appear in focus.
(3) In an implementation of this embodied procedure, the user then adjusts the Cylindrical Optical Module of the apparatus to add cylindrical power to the system. The user stops adjusting the Cylindrical Optical Module when all lines along all axes are equally blurred.
(4) In an implementation of this embodied procedure, the user then adjusts the Axis Optical Module adjustment until all lines appear clear.
(5) The user then observes the reported spherical, cylindrical, and axis refraction values that the invention shows to the user.

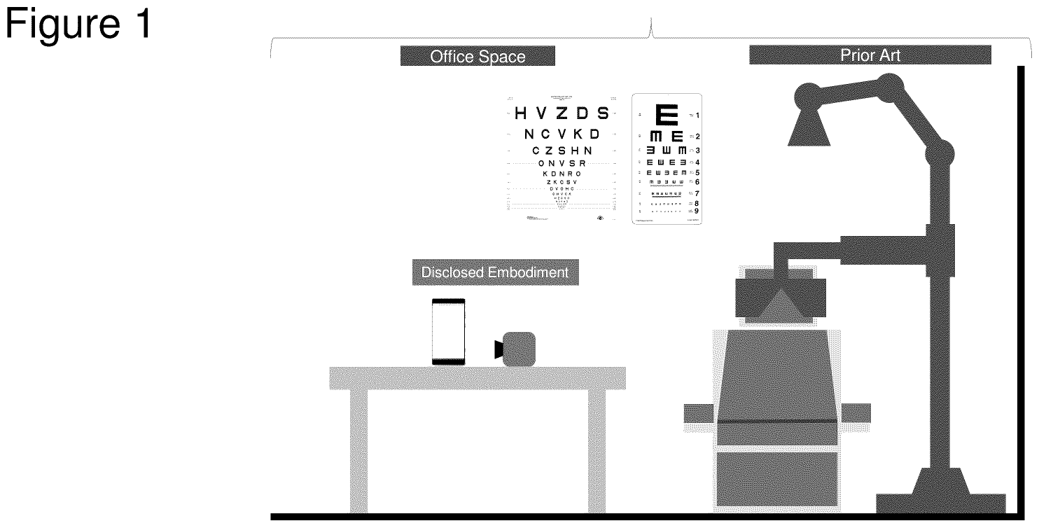

FIG. 1 depicts an illustration of a typical office layout at an optometrist office showing older previous technologies on the right and the Disclosed Embodiment on the left showcasing the benefits of the smaller form factor mobile technology for ease of use and portability.

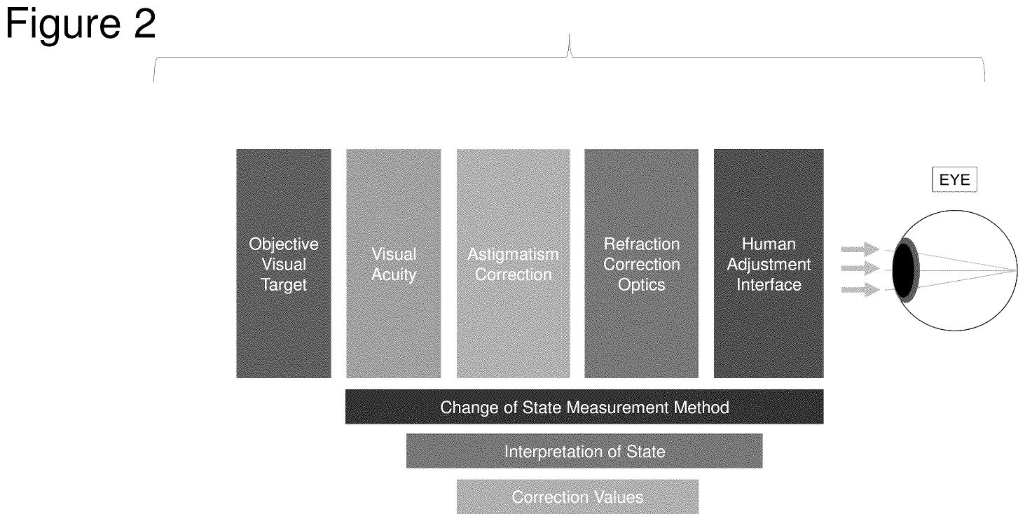

FIG. 2 shows a block diagram of the EyeQue Invention technology which may exhibit modularity in its implementation, combining several optometric evaluation techniques into one apparatus. The technology would add a layer of interpretation and evaluation to provide the end user their refractive correction values.

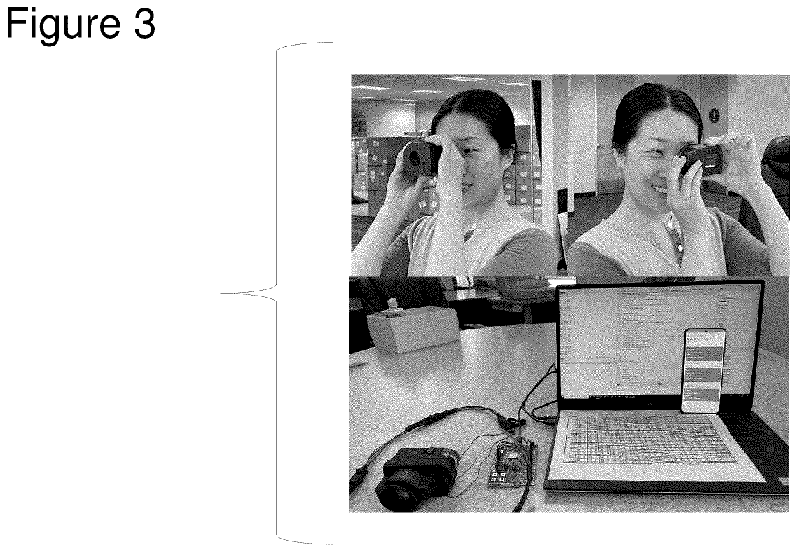

FIG. 3 depicts three possible embodiments of monocular (single optical channel) refractive measurement EyeQue invention, in concept prototype form, being operated by a user to correct their refractive need. The third embodiment depicts a possible form of data flow from a monocular version of the apparatus to a mobile device for correction values to be displayed for the user.

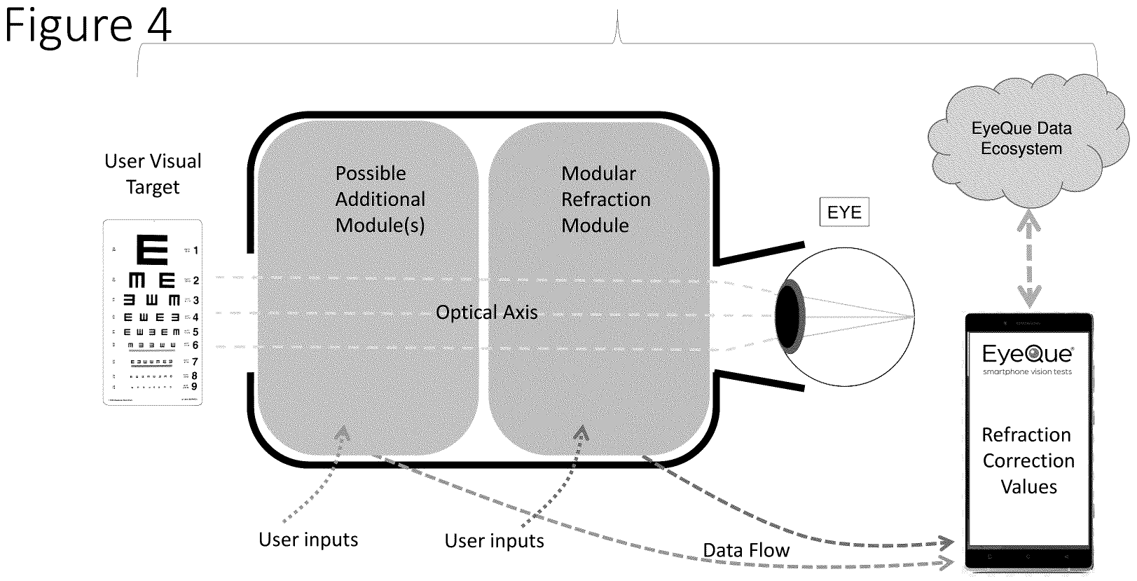

FIG. 4 depicts an embodiment of the EyeQue Apparatus with user inputs, optical inputs, and light through the apparatus and the possible modules the light may pass through, into data flow to a computational device, which displays refraction correction values to the user. This embodiment represents a monocular version, of which multiple instances of this embodiment can used use in the Invention to add better features.

FIG. 5 is a paraxial diagram depicting the concept of applying optical power to a system by varying the distance between two lenses. The power of the first lens is Ø1, the power of the second lens is Ø2, the separation between the lenses is t in a medium with index of refraction n (this is 1 when the lenses are in air). The total power of the system is described by the equation Ø total=Ø1+Ø2−Ø1 Ø2 tn. Depending on the values of Ø1, Ø2 and t, the total power in the system can be negative, 0, or positive. This concept can be used to apply spherical power when using spherical lenses, or cylindrical power when using cylindrical lenses. The lenses can be traditional lenses or Fresnel lenses.

FIG. 6 is a depiction of a deformable, or liquid lens. The lens contains a refracting liquid sack that changes its curvature as electrical current is applied, allowing the production of negative (A), 0 (B) or positive (C) optical power. This can be either a spherical or a cylindrical lens.

FIG. 7 is a depiction of a filter wheel containing lenses of varying powers. The lenses can be spherical or cylindrical.

FIG. 8 depicts the concept of a Stokes pair. Two cylindrical lenses with equal and opposite powers are placed back-to-back. As the lenses are rotated with respect to each other, the resulting cylindrical power changes.

FIG. 9 depicts the use of a Badal lens. The lens is placed such that it is one focal length away from the eye, and a target is placed one focal length behind the lens. As the target is shifted forward or backward, the vergence of the light after the Badal lens changes. The target location can be shifted until the user views the target to be in focus, and the shift distance corresponds to the refractive error of the user. The Badal lens can be spherical or cylindrical.

FIG. 10 depicts the steps for user testing with the current manifestation of the device. The image first appears blurry (A). The user adjusts the spherical module until a line spread along their astigmatic axis becomes clear (B). The user adjusts the cylindrical module until all lines become equally blurred (C). The user adjusts axis until all lines become clear (D). The dotted red circle indicates where the user should look, due to off-axis aberrations present in the current manifestation of the device.

FIG. 11 depicts a section view of a rack & pinion mechanism used to provide motion to the Spherical Optical Mechanism. This implementation consists of a spherical lens holder, two gears, a gear adapter (c), a rotary encoder (d), a user interface knob (e), and a housing (f). The spherical lens holder has two merged racks in which two pinion gears can interact. One pinion is connected to the rotary encoder while the opposite end has a pinion connected to the user interface knob for the user to rotate.

FIG. 12 depicts a section view of a rack & pinion mechanism used to provide motion to the Spherical Optical Mechanism. This implementation consists of a spherical lens holder (a), one gear (b), a gear adapter (c), a rotary encoder (d), and a user interface knob (e). The spherical lens holder has a merged racks in which two pinion gears can interact. The pinion is connected directly to the rotary encoder and the user interface knob for the user to rotate.

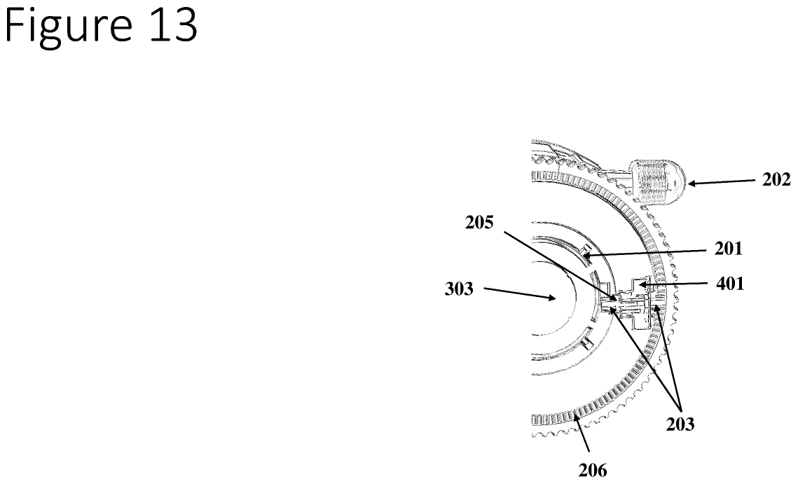

FIG. 13 depicts a section view of a rack & pinion mechanism used to provide motion to the Spherical Optical Mechanism. This implementation consists of a spherical lens holder (a), two gears (b), a rotary encoder (c), a gear adapter (d), a user interface dial (e). The spherical lens holder has a racks in which the two pinion gears can interact. One pinion is connected to the rotary encoder while the opposite end has a pinion connected to the user interface dial for the user to rotate.

FIG. 14 depicts a “Lead Screw” motion mechanism to convert rotational motion to linear motion and translate the negative spherical power lens about the optical axis. This is accomplished with means of a lens holder (a) held within a threaded barrel (b) that rotates about a fixed “rail” (c). The action of rotation on the barrel causes linear motion of the negative spherical lens (d) through the spiral thread (e) and interface of the holder (f). This negative lens translates along the shared optical axis with the positive spherical lens (g) which is fixed at one end of travel distance.

FIG. 15 depicts a section view of a Cylindrical Optical Mechanism consisting of a cylindrical lens holder (a), two gears (b), a gear rack (c), an adapter (d), rotary encoder (e), cylindrical user interface knob (f), slip ring assembly for encoder signal transmission, and outer main housing (g).

FIG. 16 depicts a view of a Cylindrical Optical Mechanism consisting of a cylindrical lens holder (a), three gears (b), an adapter (c), rotary encoder (d), cylindrical user interface knob (e), slip ring assembly for encoder signal transmission (f), and outer main housing (g).

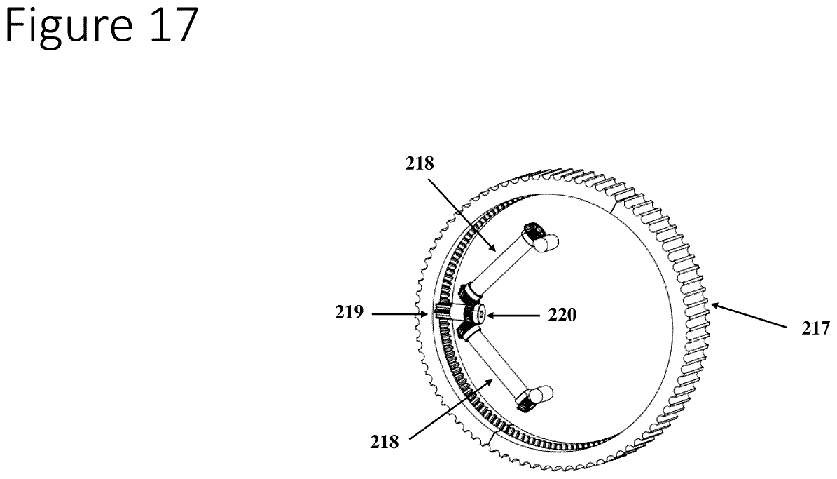

FIG. 17 depicts a view of a Cylindrical Optical Mechanism consisting of, a 45 deg bevel gear (a), two beveled gearshaft lengths (b), two bevel gear to rack interface (c), a bevel gear to dial interface (d), and a user interface dial (e).

FIG. 18 depicts a section view of a Cylindrical Optical Mechanism which uses a “Lead Screw” motion to convert rotational motion to linear motion and translate the negative cylinder power lens about the optical axis. This is accomplished with means of a lens holder (a) held within a threaded barrel (b) that rotates about a fixed “rail” (c). The action of rotation on the barrel causes linear motion of the negative cylinder lens (d) through the spiral thread (e) and interface of the holder. This negative lens translates along the shared optical axis with the positive cylinder lens (f) which is fixed at one end of travel distance.

FIG. 19 consists of a user interface rotation dial (a), two gears (b), a gear rotary encoder adapter (c), a rotary encoder (d), optical lens (e) and an electronics slip ring (f). The user rotates the large rotation dial which moves the gears and subsequent encoder which is housed within the Cylinder Optical Module. The Slip ring allows the Axis Optical Module to rotate and still transmit signal during rotation.

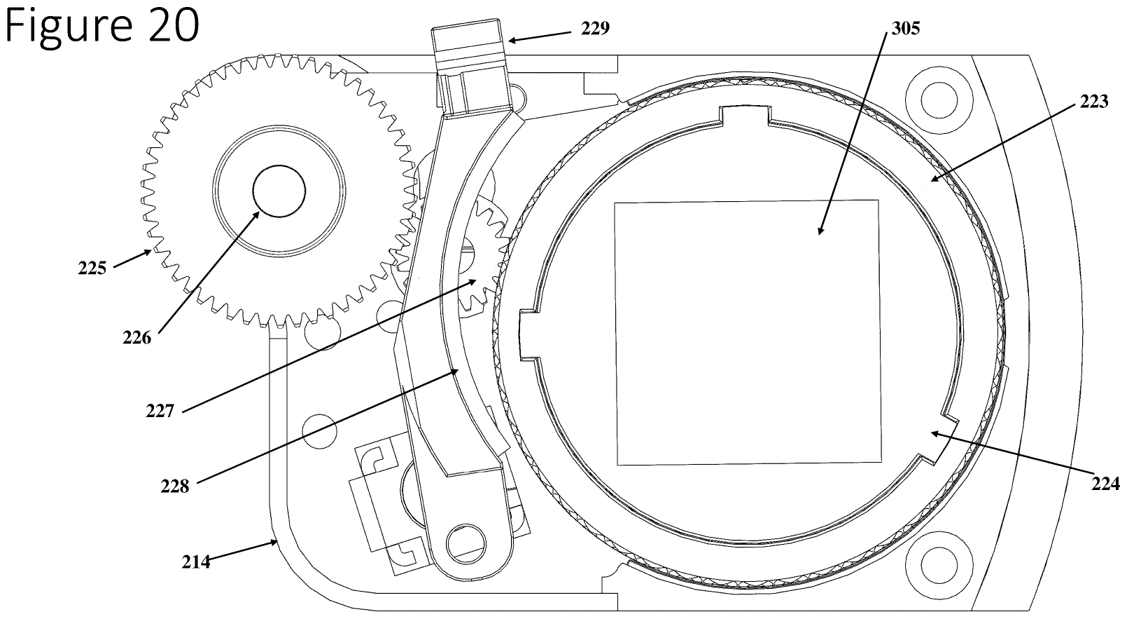

FIG. 20 depicts an Axial Optical Mechanism combined with a Cylinder Optical Mechanism. This Axial Optical Mechanism allows the entire Cylinder Optical Mechanism to rotate about the optical axis within a holder. This is done via a geartrain (a), which causes the Cylinder Optical Mechanism (b) to rotate about a holder (c). The Axial mechanism applies a stopping force to the cylinder assembly via a brake mechanism (d) when only Cylinder Optical Mechanism motion of the cylinder lenses is needed. This combined mechanism uses the fundamentals of lead screw linear motion, and clever placement of stopping forces, to be able to switch between linear motion and rotational motion. This breaking mechanism can be accomplished via friction, magnetism, fluid control, electromagnets, interlocking componentry, hard stops, etc.

FIG. 21 depicts two possible implementation of motion for the invention. The dial showcases cleverly placed figure grooves for a user to interface with as an example of human powered motion. The motor and gear (b) showcases one instance of powered motion control in which the gear in red would interface with the blue housing. Both methods would result in motion of the object as intended.

FIG. 22 depicts an implementation of a phone centering mechanism utilizing a dual spring force centering mechanism. This mechanism, when activated by pressing button (b) releases the springs (a) on either side of a phone to then push the device into place. A user then closes the housing by closing the lid (c).

FIG. 23 depicts a section and a whole view of a spring-loaded pedestal mechanism which uses friction forces and spring forces to enable a user to place and position a phone on the pedestal that stays in location after the user releases their hand. The mechanism consists of a pedestal (a), an adhesion pad for the phone (b), a housing for alignment (c) and a holder for springs (d).

FIG. 24 depicts a simple box phone holder mechanism in which a user places their cell phone into this box with a snap-to-close (a) lid. The phone screen is visible through the window and the phone will be centered via software.

FIG. 25 depicts an implementation of a monocular device (a) and of binocularity in (b) in which any number of optical channels can be present in order to accomplish the need of providing refraction correction measurement.

FIG. 26 depicts a mechanism utilizing a rack and pinion (a) combined with an outer housing (b) to provide binocularity adjustment for different users for two optical channels (c).

FIG. 27 depicts an implementation of a hinged PD mechanism on binocular implementation of the invention where a central hinge (a) is used hold together two Optical Channels (b) which pivot about that central point. Within this central point methods of measuring position electronically can be incorporated into the hinge such as a rotary encoder (c).

FIG. 28 depicts a PD mechanism which uses a single rack and pinion mechanism with a locking feature. In this mechanism only one side of the mechanism (a) moves with respect the other fixed side (b). A locking feature (c) is used to keep the PD where the user desires.

FIG. 29 depicts a similar rack and pinion mechanism which is of a more compact design. A user pulls on the tabs (a) to adjust to desired PD in which the rack and pinion (c) allow for metered movement. A set screw or button mechanism (b) then applies a locking force to keep at the desired PD.

FIG. 30 depicts a dual “4-bar linkage” system to provide PD adjustment while keeping the optical channel's orientation level with the user. One “4-bar linkage” mechanism per optical channel in a binocular system. It actuates through application of physical linkages (a) and sliding joints (b) in the assembly of a housing to maintain level orientation of the optical channel it is housing. This form of PD adjustment can have each set of linkages freely moving with respect to themselves or they can be connected via powertrain means (c) so that the motion of one side is mirrored by the other. This mechanism can be done with other “N-bar linkage” systems or with other types of mechanical connection during binocularity. This mechanism is not limited to a singular type of powertrain connection should it be implemented, and several options are available for use such as geartrain, belt drive, friction drive, and more not listed.

FIG. 31 depicts a schematic of modularity of function and form. A modular device consisting of possibly many compounded mechanism (a) will need to connected to itself via some means electronically, mechanically, and optically (b) such that the user can obtain the refraction correction that they desire for their eye (c).

FIG. 32 depicts a modular mechanism consisting of lip (a) and groove (b) features, snap fit features (c) & (d), and a central pin alignment feature to form a hinge (e).

FIG. 33 depicts a section view of the invention consisting of two modules which have alignment pin features and that utilize magnetic attachment (a) & (b) to keep the modules together.

FIG. 34 depicts a section view of two modules held together via a spring-loaded button mechanism which keeps modules attached to one another. A spring (a) within the mechanism keeps constant clamping force on the latch (b) which pivots about a fulcrum. The latch is released when a user presses a button (c).

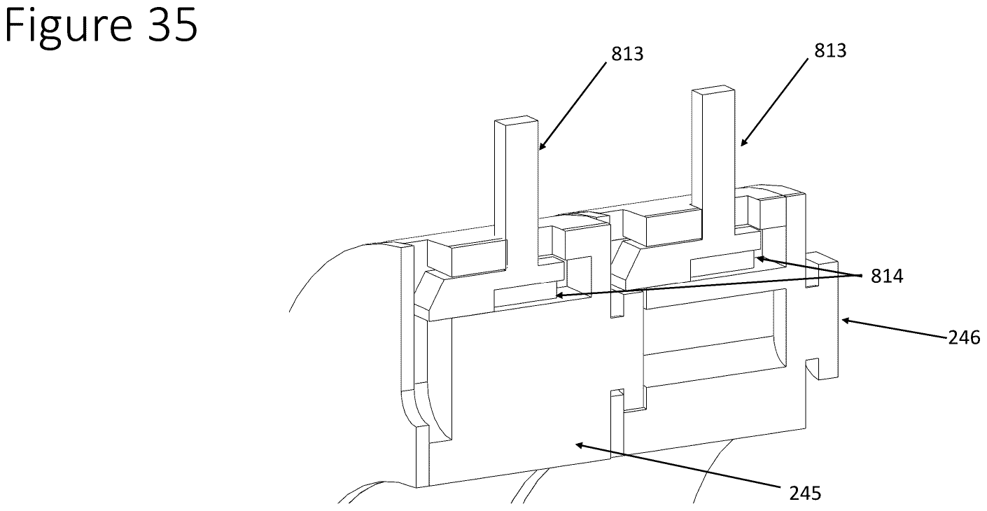

FIG. 35 depicts a section view of two modules held together via a spring-loaded slide mechanism which keeps modules attached to one another. A slide (a) within the mechanism is held close via a spring (b) keeping the modules together. A user pushes on the slide to dis-engage the lock release the module.

FIG. 36 depicts a section and a whole view of an interlocking axle mechanism which has a “twist to lock” action to combine multiple pins together, enabling modularity. This mechanism accomplishes this with a twist lock interface male side (a) which is matched with a corresponding interface female side (b) on each instance of the pin.

FIG. 37 depicts a diagram of a user (a) needing to center the EyeQue invention (b) to the center of their eye's optical path (c). This can be done any number of ways through mechanism which hold the invention, or the user, or means powered or non-powered forms of centering the invention to the uer's eye in monocular, binocular, or other forms.

FIG. 38 depicts three vies of an eyecup which can be integrated with the invention to cover the region around the user's eyeball to prevent stay light.

FIG. 39 depicts two views of a facemask mechanism which has integrated eyecups (a) which block stray light from entering the user's FOV while also having a hinged PD mechanism (b) integrated within the device such that the PD can be adjust at the same time. This facemask implementation asl has modularity integration which interlocking features (c) on the back.

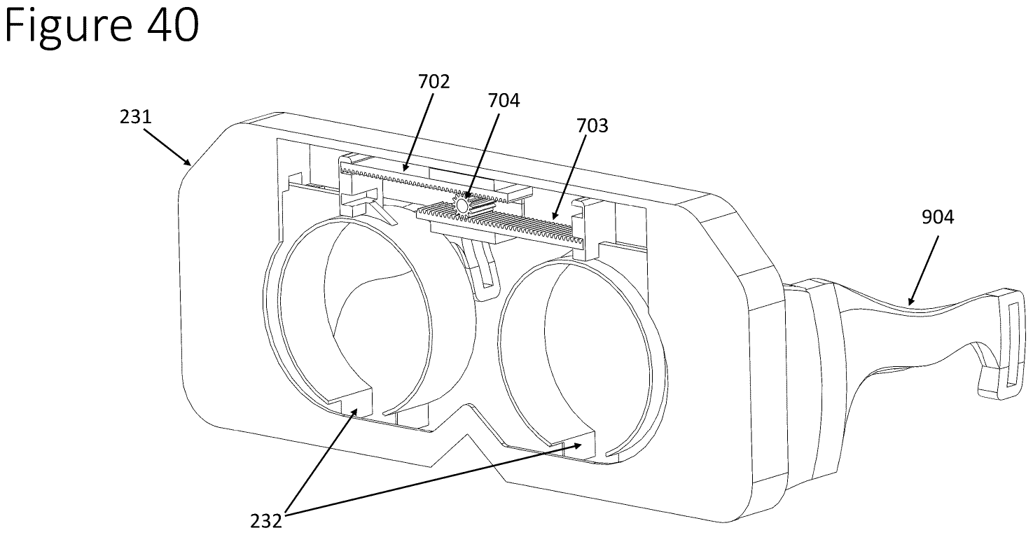

FIG. 40 shows a facemask implementation which utilizes an eyeglasses type holder (a) approach the design combined with a rigid structure (b) which supports a rack an pinion PD mechanism (c) that can adjust the two optical channels (d) in the binocular mechanism

FIG. 41 shows a singular generic foam block facemask for a one-size-fits-all type approach consisting of the single block (a), cutout features for a user's face (b) and attachment locations for PD mechanism and subsequent modules of the invention (c).

FIG. 42 shows a block diagram of the electronics and data flow from the user inputs, motion sensing solutions, and status indicators through to the central processing unit of the invention. The central processing unit sends this data via a wireless module to a smart device running EyeQue developed software which with optional connectivity to additional capabilities for data processing via EyeQue's cloud ecosystem.

FIG. 43 shows a diagram of a possible implementation of change of state sensing through encoders (a) and electronics board (b) which send data via wireless means (c) to a smart device (d) for showing to the user.

FIG. 44 is a Zemax OpticStudio lens diagram for the current manifestation of the device optical system depicting the lenses comprising the cylindrical module, the lenses comprising the spherical module and a paraxial model of the human eye.

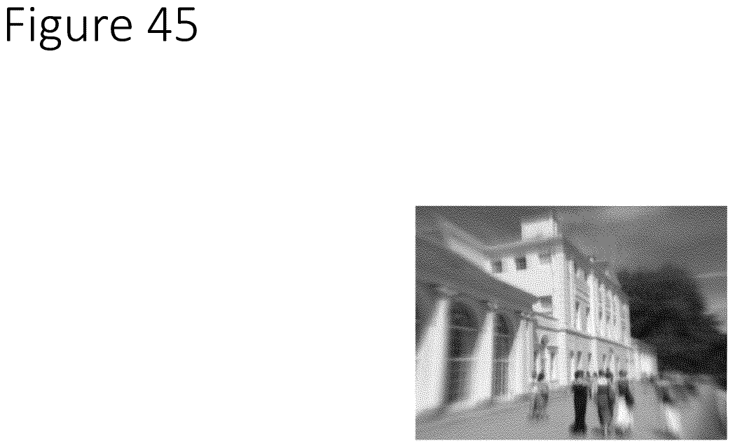

FIG. 45 is a Zemax OpticStudio image simulation depicting the current manifestation of the device with a FOV (Field of View) of 15o.

FIG. 46 is a Zemax OpticStudio lens diagram for an alternate manifestation of the device optical system depicting (A) the lenses comprising the cylindrical module, (B) the lenses comprising the spherical module and (C) a paraxial model of the human eye.

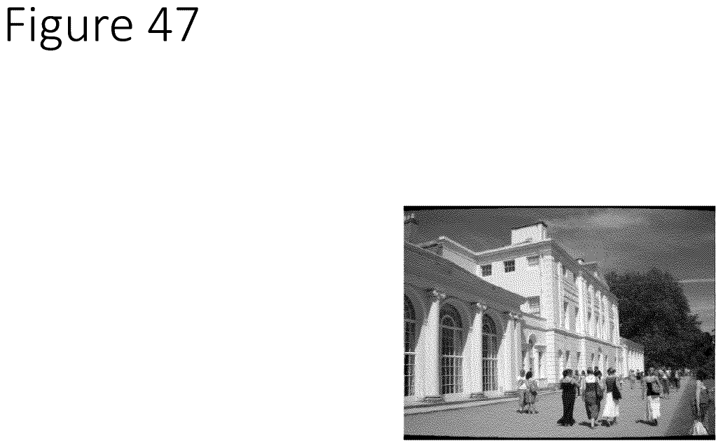

FIG. 47 is a Zemax OpticStudio image simulation depicting an alternate manifestation of the device with a FOV of 30o.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

Any and all the above references and U.S. patents and applications are incorporated herein by reference. Aspects of the invention can be modified, if necessary, to employ the systems, functions and concepts of the various patents and applications described above to provide yet further embodiments of the invention.

### Items

The following items may be used as the basis for claims in related non-provisional patent applications.

A method [FIG. 10] of using a spherical optical mechanism by an optical system (user) the method comprising the steps of:
a) producing a blurry image;
b) adjusting a spherical module until a line spread along the optical systems astigmatic axis become clear to the optical system;
c) adjusting a cylinder module until all lines become equally blurred to the optical system; and
d) adjusting an axis until all lines become clear to the optical system.

The method of 1, further including the step of using the spherical optical system to product a circle directing the optical system's view, the step used to offset off-axis aberrations of the spherical optical system.

The method of 1 using a first lens (300) and a second lens (301) (FIG. 5.

The method of 1 further including the steps of using a rack and pinon mechanism (FIG. 11) to move the first and second lenses of the system, the rack and pinon mechanism comprising a spherical lens holder, two gears, a gear adapter, a rotary encode, a user interface knob, and a housing.

The method of 4 wherein the spherical lens holder uses two merged racks in which two pinion gears may interact.

The method of 1 using a (FIG. 12) a rack & pinion mechanism to provide lens movement to the spherical optical mechanism by using a spherical lens holder, one gear, a gear adapter, a rotary encoder, and a user interface knob.

The method of 6 wherein the spherical lens holder uses merged racks in which two pinion gears can interact.

The method of 7 using the pinion connected directly to the rotary encoder and the user interface knob for the user to rotate.

The method of 1 using a [FIG. 13] a rack and pinion mechanism used to provide motion to the lenses of the spherical optical mechanism, using a spherical lens holder, two gears, a rotary encoder, a gear adapter, a user interface dial.

the method of 9 wherein spherical lens holder has a racks in which the two pinion gears can interact.

The method of 10 using one pinion connected to the rotary encoder while the opposite end uses a pinion connected to the user interface dial for the user to rotate.

The method of 1 using a lead screw (FIG. 14) motion mechanism to convert rotational motion to linear motion and translate the negative spherical power lens about the optical axis.

The method of 12 using a lens holder held within a threaded barrel that rotates about a fixed rail.

The method of 13 using the action of rotation on the barrel to cause linear motion of the negative spherical lens through the spiral thread and interface of the holder.

The method of 14 further including the step of using the negative lens to translate along the shared optical axis with the positive spherical lens which is fixed at one end of travel distance.

The method of 1 using a cylindrical optical mechanism (FIG. 15) comprising a cylindrical lens holder, two gears, a gear rack, an adapter, rotary encoder, cylindrical user interface knob, slip ring assembly for encoder signal transmission, and outer main housing.

The method of 1 using a cylindrical optical mechanism (FIG. 16) comprising of a cylindrical lens holder, three gears, an adapter, rotary encoder, cylindrical user interface knob, slip ring assembly for encoder signal transmission, and outer main housing.

The method of 1 using a a cylindrical optical mechanism (FIG. 17) comprising a 45 deg bevel gear , two beveled gearshaft rods, two bevel gear to rack interface, a bevel gear to dial interface, and a user interface dial.

The method of 1 wherein a cylindrical optical mechanism uses a lead screw motion (FIG. 18) to convert rotational motion to linear motion and translate the negative cylinder power lens about the optical axis.

The method of 19 using a lens holder held within a threaded barrel that rotates about a fixed rail.

The method of 20 using action of rotation on the barrel to cause linear motion of the negative cylinder lens through the spiral thread and interface of the holder.

The method of 1 having a user interface (FIG. 19) using a rotation dial, two gears, a gear rotary encoder adapter, a rotary encoder, optical lens and an electronics slip ring.

The method of 22 further including the step of rotating the large rotation dial which moves the gears and subsequent encoder which is housed within the cylinder optical module.

The method of 23 using the slip ring to allow the axis optical module to rotate and still transmit signal during rotation.

The method of 1 further including the step of combining an axial optical system with a cylinder optical mechanism, (FIG. 20) the axial optical mechanism allowing the cylinder optical mechanism to rotate about the optical axis within a holder.

The method of 25 further including the step of using a geartrain to causes the cylinder optical mechanism to rotate about the holder.

The method of 26 further including the step of using the axial mechanism to apply stopping force to the cylinder assembly by use of a break mechanism when cylinder optical mechanism motion of the cylinder lenses is needed.

The method of 27 further including the steps of using lead screw linear motion, placement of stopping forces disposed within the system, to be able to switch between linear motion and rotational motion.

The method of 1 including the step of using (FIG. 26) a mechanism utilizing a rack and pinion combined with an outer housing to provide binocularity adjustment for different users for two optical channels.

The method of 1 including the step of using a hinged pupillary distance mechanism (FIG. 27) on binocular implementation wherein a central hinge is used hold together two optical channels which pivot about that central point.

The method of 30 wherein a central point method of measuring position electronically is incorporated into the hinge such as a rotary encoder.

The method of 1 further including the step of using a rack and pinion mechanism (FIG. 29a b) wherein a user pulls on the tabs to adjust to a desired PD in which the rack and pinion allows for metered movement and wherein a set screw or button mechanism then applies a locking force to retain the desired PD.

The method of 1 further including the steps of (FIG. 31) using a plurality of modules comprising an objective visual target mechanism, visual acuity mechanism, axial optical mechanism, cylindrical optical mechanism and spherical optical mechanism in visual connection with a measured optical system.

The method of 33 further including the step of electronically connecting the plurality of modules to a personal electronic device for instant data readings.

The method of 33 further including the step of securing two or more modules together by use of alignment pines and magnetic attachment.

The method of 33 further including the step (FIG. 34) of securing two or more modules together by use of a spring-loaded button mechanism wherein a spring within the mechanism applies constant clamping force to a latch which pivots about a fulcrum.

The method of 33 further including the step of securing two or more modules (FIG. 35) by use of a spring loaded slide mechanism with the slide disposed within the mechanism is urged in a closed position by force of the spring or other elastic member.

The method of 33 further including the step of securing two or more modules (FIG. 36) by use of an interlocking axle mechanism comprising a twist to lock configuration to combine multiple pins together, enabling modularity.

The method of 38 further comprising the step of using a twist lock interface male side which mechanically comports with a corresponding interface female side on each instance of the pin.

The method of further including the step of using (FIG. 43) a change of state system gathering system data by use of encoders in electronic attachment to a an electronics board with the electronics board transmitting data to a personal electronic device displaying data to an end user.

### Reserved

What is claimed is:

1. A method [FIG. 10] of using a spherical optical mechanism of an optical system the method comprising the steps of:
a) producing a blurry image;
b) adjusting a spherical module until a line spread along the optical system's astigmatic axis become clear to the optical system;
c) adjusting a cylinder module until all lines become equally blurred to the optical system; and
d) adjusting an axis until all lines become clear to the optical system.

2. The method of claim 1, further including the step of using the spherical optical system to product a circle directing the optical system's view, the step used to offset off-axis aberrations of the spherical optical system.

3. The method of claim 1 using a first lens (300) and a second lens (301) (FIG. 5 to produce the blurry image.

4. The method of claim 3 further including the steps of using a rack and pinon mechanism (FIG. 11) to move the first and second lenses of the system, the rack and pinon mechanism comprising a spherical lens holder, two gears, a gear adapter, a rotary encode, a user interface knob, and a housing.

5. The method of claim 4 wherein the spherical lens holder uses two merged racks in which two pinion gears may interact.

6. The method of claim 2 using a (FIG. 12) a rack & pinion mechanism to provide lens movement to the spherical optical mechanism by using a spherical lens holder, one gear, a gear adapter, a rotary encoder, and a user interface knob.

7. The method of 6 wherein the spherical lens holder uses merged racks in which two pinion gears can interact.

8. The method of claim 7 using the pinion connected directly to the rotary encoder and the user interface knob for the user to rotate.

9. The method of claim 2 using a [FIG. 13] a rack and pinion mechanism used to provide motion to the lenses of the spherical optical mechanism, using a spherical lens holder, two gears, a rotary encoder, a gear adapter and a user interface dial.

10. The method of claim 9 wherein spherical lens holder has a racks in which the two pinion gears can interact.

11. The method of claim 10 using one pinion connected to the rotary encoder while the opposite end uses a pinion connected to the user interface dial for the user to rotate.
